(19) United States
(12) Patent Application Publication
(10) Pub. No.: US 2007/0178068 A1
(43) Pub. Date: Aug. 2, 2007

(54) COMPOSITIONS AND METHODS FOR REGULATING COMPLEMENT SYSTEM

(76) Inventors: Samuel Jotham Reich, Merion Station, PA (US); Nadine Dejneka, Wynnewood, PA (US)

Correspondence Address: PEPPER HAMILTON LLP, ONE MELLON CENTER, 50TH FLOOR, 500 GRANT STREET, PITTSBURGH, PA, 15219, US

(21) Appl. No.: 11/615,554

(22) Filed: Dec. 22, 2006

Related U.S. Application Data
(60) Provisional application No. 60/753,041, filed on Dec. 22, 2005.

(51) Int. Cl. A61K 48/00; C07H 21/02; A61K 9/127

(52) U.S. Cl. 424/93.2; 514/44; 514/81; 424/450; 536/23.1

(57) ABSTRACT

The disclosure provided herein provides for methods and compositions for modulating the human complement pathway by reducing the expression or production of one or more complement pathway protein and uses for such methods for treating diseases including ocular disease and macular degeneration related disease. The invention also includes active agents for mediating the modulation of the complement pathway including siRNA designed to cause the RNAi-mediated degradation of complement pathway proteins.

### CROSS REFERENCE

The application claims priority from U.S. Provisional Application No. 60/753,041 filed Dec. 22, 2005, herein incorporated by reference in its entirety.

### BACKGROUND

The complement system includes some 30 proteins that circulate in the extracellular fluid capable of launching a non-antigen specific immune response against invading bacterial and viral pathogens. Complement system proteins are circulated in an inactive form and become activated when a molecular pattern on the surface of an invading microorganism is recognized by early components of the system. The activated early components of the complement system recruit other complement proteins to the surface of the invader forming activated complexes that act as proteinases proteolytically cleaving other members of the complement system and activating them. What follows is a cascade of proteolytic activation, recruitment, and co-binding of members of the complement system resulting in the formation of the membrane attack complex (MAC), a pore in the cell wall of the invader resulting in cytolysis.

The complement system is activated by three different pathways: the classical pathway, the alternative pathway, and the lectin pathway. Each pathway culminates in the formation of the MAC but is initiated by a different stimulus.

The classical pathway is initiated when antibody (multiple IgG molecules or a single IgM molecule) binds to the surface of the pathogen. Binding allows the Fc component of the antibody to interact with the C1q subunit of factor C1 leading to the activation of subunit C1r. C1r, a proteolytic enzyme, cleaves subunit C1s and activates its protease function. C1s cleaves factor C4 to generate C4a and C4b. C4a remains biologically active at the reaction site mediating inflammation. C4b can either become an inactive by-product, bond covalently with an IgG molecule, or covalently attach to the cell surface of the antibody-bound microbe. In the presence of Mg⁺², cell surface bound C4b recruits C2 to the cell surface where it is cleaved by C1s to generate C2b and C2a. C2b diffuses from the cell surface. C2a forms a complex with C4b to form C3 convertase (C4b2a), a C3 specific protease.

C3 convertase cleaves C3 to generate C3a and C3b fragments. C3a is a potent anaphylatoxin that diffuses away from the cell surface and acts as a chemoattractant for leukocytes. Like C4b, C3b can become an inactive by-product, but alternatively, up to 10% of C3b can remain covalently bound to the plasma membrane of the pathogen. While bound to the plasma membrane C3b can serve as an opsonin or can combine with C3 convertase (C4b2a) to form C5 convertase (C4b2a3b). C5 convertase binds to and cleaves C5 generating C5a and C5b. C5a diffuses away from the cell surface and acts as a potent anaphylatoxin and chemoattractant that mediates inflammation. C5b remains bound to the cell wall of the pathogen and initiates the assembly of the MAC.

The MAC is formed from the late components of the complement system. C5b bound to the cell wall of the pathogen recruits C6 to form a C5bC6 complex that, in turn, recruits C7 to form a C5bC67 complex that inserts into the cell membrane of the pathogen. C8 then binds to C5b67 forming a C5b678 complex and this complex initiates C9 polymerization resulting in the insertion of C9 into the lipid bilayer of the pathogen and the formation of the MAC. The MAC creates a pore in the cell membrane of the pathogen resulting in an influx of small molecules, ions and water into the cell ultimately leading to osmotic cell lysis.

Activation of the lectin pathway is based on carbohydrate recognition. Mannose binding lectin (MBL) and ficolins, which structurally resemble C1q, bind to specific carbohydrates on the pathogen cell surface and associate with MBL-associated serine proteases (MASPs), which resemble C1r and C1s. MASP-2, like C1s, cleaves complement components C4 and C2 initiating the formation C3 convertase. MASP-1 stimulates the alternative pathway by cleaving C3 directly. Cleavage of C3 results in the formation of C5 convertase and eventually MAC formation as described above.

The alternative pathway is activated in the absence of antibody binding by low-grade cleavage of C3 in plasma. A C3b produced in the plasma covalently binds to hydroxyl groups on cell-surface carbohydrates and proteins. C3b bound to the cell surface of the pathogen recruits Factor B to the cell surface. Factor D, which is mainly produced by adipose cells, cleaves bound Factor B to generate Ba and Bb. Ba is released into the circulation. Bb binds C3b to form the alternative pathway C3 convertase, C3bBb. Properdin stabilizes C3 convertase and allows the complex to continue to cleave C3. A proportion of the C3b produced binds C3bBb forming the alternative pathway C5 convertase, C3bBb3b. Like the other C5 convertases, alternative pathway C5 convertase cleaves C5 initiating MAC assembly.

Receptor binding mediates the activation of several complement system components. iC3b, a fragment generated by Factor I-mediated cleavage of C3b, and C4b bind complement receptor type 1 (CR1, CD35) with high affinity. CR1 is an integral membrane protein found on erythrocytes, macrophages, monocytes, polymorphonuclear leukocytes, B cells and follicular dendritic cells that promotes phagocytosis of C3b- and C4b-coated particles and mediates the clearance of immune complexes from the circulation. Complement receptor type 2 (CR2, C3d receptor, CD21), a membrane glycoprotein found on B lymphocytes, follicular dentritic cells and epithelial cells, specifically binds the cleavage product of Factor I mediated C3b cleavage. Complement receptor type 3 (CR3, MAC1, CD11bCD18) is found on macrophages, monocytes, polymorphonuclear leukocytes and dentritic cells and is thought to stimulate the phagocytosis of iC3b-coated microorganisms and particles by binding iC3b. Complement receptor type 4 (CR4, p150,95, CD11cCD18) binds iC3b as well as C3dg (another cleavage product of C3b) and promotes phagocytosis. Its cellular distribution is similar to that of CR3. Finally, C5a receptor (C5aR) and the C3a/4a receptor are found on endothelial cells, mast cells and phagocytes and bind C5a and C3a or C4a, respectively, to promote the anaphylatoxic and chemotactic activities of these protein fragments.

The complement system is tightly regulated to avoid erroneous activation or immune attack on host cells. The classical pathway is regulated by at least 6 different proteins. C1 esterase inhibitor (C1 INH) combines with and inactivates C1r and C1s as well as MASP-1 and MASP-2 proteases of the lectin pathway to inhibit the cleavage of C2 and C4 and the formation of C3 convertase. Most of the C1 found in blood is bound to C1 INH preventing spontaneous activation; however C1 is released from C1 INH when bound to an antigen-antibody complex activating the classical pathway. Another regulator, Factor I of the classical pathway, proteolitically cleaves C4b and C4b binding protein (C4bBP) inactivates Factor I using CR1 or membrane cofactor protein (MCP, CD46) as cofactors. In addition to serving as cofactors for Factor I, C4bBP and CR1 bind to C4b and competitively inhibit the binding of C2a, which inhibits the production of classical pathway C3 convertase by promoting its dissociation. Decay accelerating factor (DAF) a transmembrane glycoprotein found on peripheral blood cells, endothelium, and some mucosal epithelial cells, promotes the dissociation of classical pathway C3 convertase and binds C4b to prevent it from binding C2.

The alternative pathway is also tightly regulated. Factor H competes with Factor B and Bb for the binding of C3b inhibiting production of alternative pathway C3 convertase. Factor B binding is favored on surfaces with high sialic acid content, and since most bacterial cells have low amounts of surface sialic acid compared to mammalian cells, Factor B binds bacterial cells readily activating of the complement system. In contrast, Factor H binds polyanionic molecules like glycosaminoglycans or sulphated polysaccharides such as heparin that are located on the surface of mammalian cells. Factor H, therefore, binds mammalian cells protecting them from the complement system. Additionally, MCP and CR1 increase the affinity of surface bound C3b for Factor H and inactivates alternative pathway C3 convertase by promoting the dissociation of Bb. Factor I has a similar effect on alternative pathway C3 convertase using Factor H, CR1 and MCP as cofactors.

Complement regulation also occurs at the MAC. C5b67 complex insertion into the lipid membrane is inhibited by vitronectin (S-protein) binding in the serum, and membrane inhibitor of reactive lysis (CD 59, MRL) and homologous restriction factor (HRF) are membrane bound proteins found on erythrocytes, lymphocytes, monocytes, neutrophils, and platelets that prevent C7 and C8 from binding to C5b6 and interfere with C9 binding to C8, respectively. Finally, clusterin (SP-40/40), another serum protein, modulates MAC formation by preventing C9 assembly on C5b-8 and C5b-9 and by preventing bound C5b67 from attaching to the membrane.

Complement deficiencies also promote disease by increasing susceptibility to bacterial and yeast infections. Patients with defects in antibody production or phagocytic function, or have defective classical pathway complement proteins are at high risk for developing Haemophilus influenzae and Streptococcus pneumoniae infections. Similarly, patients with inherited deficiencies in components of the MAC are susceptible to neisserial disease, especially Neisseria meningitides, and reduced levels of MBL have been linked to recurrent pyogenic (pus forming) infections and a failure to thrive in young children. Interestingly, adults with a similar opsonin deficiency are healthy suggesting that the MBL pathway plays a critical role during the period when passively acquired maternal antibodies are lost and the mature immune system is being developed.

Many microorganisms have taken advantage of the complement system to avoid host mediated immune response and promote their virulence. Several pathogens bind C4bBP and/or Factor H. Epstein-Barr virus envelope glycoprotein gp350/220 binds CR2, and human immunodeficiency virus (HIV) and pathogenic mycobacteria bind C3b and use C3 receptors to gain entry into the cell. Other bacteria express proteins that inhibit activation of complement, and still others have developed thick capsules that form physical barriers against MAC formation.

Viruses avoid complement by incorporating complement regulatory proteins into their envelope (i.e., HIV), producing proteins that structurally mimic complement regulator proteins, or produce proteins that do not possess structural homology to complement recognition proteins but possess similar functional properties. Vaccinia virus is of specific interest because it secretes vaccinia complement control protein (VCP), a complement inhibitor whose amino acid sequence resembles host C4bBP (38%), MCP (35%) and DAF (31%). While, VCP is most structurally similar to C4bBP, its functional profile is most similar to CR1. VCP blocks complement action at several stages of the pathway by binding to C4b or C3b. It is also able to bind heparin which may confer the ability of VCP to bind to endothelial cells and block the attachment of small chemo-attractant cytokines which regulate the localization and migration of leukocytes into the tissues. Based on these properties, VCP has a strong potential as a therapeutic agent for diseases involving aberrant complement activation. VCP blocks complement activation by β-amyloid protein making it a potentially useful treatment of Alzheimer's disease and representing a promising treatment for hyperacute rejection following xenotransplantation. Studies have shown that VCP can prolong survival of xenotransplanted organs in vivo by blocking complement activation. VCP may also be useful for the treatment of multiple organ dysfunction as well as brain and spinal cord injury.

Deficiencies in any single protein component can lead to abnormal complement activation, resulting in limited MAC formation and a lack of complement-mediated response. Additionally, aberrant activation can result in human disease, and members of the complement cascade have been implicated in the development of a diverse group of diseases for example, systemic lupus erythematosus (SLE), sepsis, immune complex disease, inflammation, pulmonary and hepatic fibrosis, asthma, atherosclerosis, diabetes and Alzheimer's disease. Abnormal stimuli, such as the presence of persistent microorganisms or autoimmune humoral responses to self antigens, can trigger the abnormal complement system activation. Thus, a major goal of research has been to identify effective agents to target the specific complement components and inactivate the pathway, and the design of novel therapeutics for complement-mediated disease.

The enzymatic cleavage of C5 initiates MAC assembly. A small molecule C5aR antagonist (AcF-OPdChaWR) has produced favorable effects in multiple in vivo models including significant anti-inflammatory activity in a rodent model of sepsis and prevention of progressive impairment of renal function and reduced infiltration of neutrophils and macrophages in a murine lupus nephritis mode. Furthermore, AcF-OPdChaWR significantly reduced neutrophil extravasation in a traumatic brain injury model, and intravenous administration of small molecule C5aR inhibitors significantly reduced liver collagen levels and fibrosis in a murine hepatic fibrogenesis model.

Another C5 inhibitor, the sodium salt of K-76 monocarboxylic acid (K-76COONa) prevents C5b generation and facilitates its decay. Oral administration of this compound to diabetic rats resulted in a reduction in proteinuria and mesangial expansion in glomeruli

C5 has also been targeted using antibodies. Pexelizumab, a monoclonal antibody, significantly reduced mortality following acute myocardial infarction (MI) when administered as adjunctive therapy with primary percutaneous coronary intervention and reduced mortality or MI in patients after coronary artery bypass graft surgery, in the presence or absence of valve surgery. Pexelizumab has completed a Phase III clinical trial (PRIMO-CABG). While the primary endpoint of the study was not achieved, there was an overall decrease in post-operative patient mortality and morbidity. Eculizumab, another monoclonal antibody, has been tested in patients with rheumatoid arthritis, idiopathic membranous nephropathy, SLE, dermatomyositis and paroxysmal nocturnal hemaglobinuria, and evidence of clinical improvement in these conditions has been observed.

C3 inhibition has also been the focus of research. N²-[2,2-diphenylethoxy)acetyl]-L-arginine (SB 290157), a low molecular weight nonpeptide antagonist of C3aR, decreased paw edema in a rodent arthritis model and inhibited neutrophil recruitment in a guinea pig airway neutrophilia model. Compstatin, a cyclic 13 residue peptide, which is species-specific for primate C3, binds C3 and inhibits complement activation in serum. Furthermore, the peptide has been shown to extend the life of a porcine-to-human xenograft perfused with human blood.

Researchers have explored the possibility of targeting Factors B and D with monoclonal antibodies to specifically inhibit the alternative pathway. mAB1379 is directed against an epitope of mouse factor B and inhibits the alternative pathway in serum of mice, rats, humans, monkeys, pigs and horses. In a murine anti-phospholipid syndrome model, an autoimmune disease characterized by recurrent fetal loss, vascular thrombosis, and thrombocytopenia in the presence of anti-phospholipid antibodies, intraperitoneal administration of the antibody provided protection from complement activation and fetal loss, suggesting a therapeutic potential for the inhibition of Factor B. 166-32 is a monoclonal antibody directed against human Factor D that successfully inhibited the activation of complement, leukocytes and platelets in a cardiopulmonary bypass model. Alternative antibodies directed against Factor D have also been effective in vitro and in vivo.

The use of native and modified complement regulatory proteins is also being explored as potential therapeutics. C1 INH has been used clinically for more than 25 years for the treatment of hereditary angioedema, an autosomal dominant condition that is caused by C1 INH deficiency, and has shown promising results in a number of other disease models including sepsis, brain and myocardial ischemia-reperfusion injury, hyperacute transplant rejection, traumatic shock and the vascular leak syndromes associated with thermal injury IL-2 therapy, and cardiopulmonary bypass. The cardio-protective effects of C1 INH have been demonstrated in human clinical trials for myocardial ischemia-reperfusion injury; however, they are dose dependent, and excess C1 INH has proven fatal to newborns during cardiopulmonary bypass.

TP10 is a soluble form of CR1 (sCR1) that has been tested in a clinical trial for cardiopulmonary bypass. While early results showed TP10 was well tolerated and beneficial, Phase II results failed to meet their primary endpoint. Nevertheless, further subgroup analysis of data from male patients in a high-risk population undergoing open-heart procedures revealed a significant decrease in infarct size and mortality, thus additional trials are planned. TP20 an alternative glycosylated form of TP10 has also been evaluated in vivo and in vitro.

APT070 (Mirococept) is a drug derived from a bacterially expressed region of CR1 combined with a membrane-targeting peptide that has been efficacious in animal models of vascular shock, rheumatoid arthritis, renal transplantation and Guillain-Barre syndrome. It has been administered systematically to healthy patients and locally to patients with rheumatoid arthritis and undergoing renal transplantation. A membrane-targeting strategy similar to that of APT070 has also been applied to CD59 and DAF.

The ability to target a complement inhibitor to a site of injury is particularly appealing as this should enhance efficacy and avoid systemic inhibition of complement. One possible strategy for targeting inhibitors was effectively demonstrated in a puromycin-induced nephrosis rat model with the use of a single chain antibody fragment. An antibody fragment specific for rat glomerular and proximal tubular epithelial cells was linked to Crry, a C3 convertase regulator found in rodents, and CD59. Intraperitoneal injection of either modified inhibitor resulted in protection against tubulointerstitial injury and renal dysfunction.

Studies have demonstrated that the presence of macular drusen is a strong risk factor for the development of both atrophic and neovascular AMD. Drusen causes a lateral stretching of the RPE monolayer and physical displacement of the RPE from its immediate vascular supply, the choriocapillaris. This displacement creates a physical barrier that may impede normal metabolite and waste diffusion between the choriocapillaris and the retina. It is likely that wastes may be concentrated near the RPE and that the diffusion of oxygen, glucose, and other nutritive or regulatory serum-associated molecules required to maintain the health of the retina and RPE are inhibited. It has also been suggested that drusen perturb photoreceptor cell function by placing pressure on rods and cones and/or by distorting photoreceptor cell alignment.

Recent studies have linked complement proteins to drusen in the eye. The presence of complement factors in drusen provides evidence that complement may play a role in AMD. Therefore, the ability to regulate complement activity may be beneficial in treating AMD.

### SUMMARY OF INVENTION

Embodiments of the invention presented herein are generally directed to isolated short interfering RNA (siRNA) having a sense of RNA strand substantially identical to a complement pathway protein mRNA or complement system regulatory protein mRNA. In some embodiments, the siRNA may include a sense RNA strand having a nucleotide sequence substantially identical to a target sequence of from about 19 to about 25 contiguous nucleotides of a complement pathway protein mRNA or complement system regulatory protein mRNA and an antisense RNA strand having a sequence substantially complementary to the sense RNA strand wherein the sense RNA strand and the antisense RNA strand form an RNA duplex. In various embodiments, the siRNA may include a sense RNA strand having a nucleotide sequence substantially identical to a target sequence of from about 19 to 25 contiguous nucleotides of human classical, alternative, and lectin complement pathway proteins, C1, C2, C3, C4, C5, C6, C7, C8, C9, Factor B, Factor D, Factor H, Factor I, or vitronectin, clusterin, C4bBP, MCP and DAF. In preferred embodiments, the siRNA may include a sense RNA strand having a nucleotide sequence substantially identical to a target sequence of from about 19 to about 25 contiguous nucleotides of human C3 protein mRNA.

In some embodiments, the sense RNA strand may be one RNA molecule, and the antisense RNA strand may be one RNA molecule, and in other embodiments, the sense and antisense RNA strands of the siRNA may be covalently linked. In certain embodiments, the sense and antisense RNA strands may be stabilized against nuclease degradation, and the siRNA may further include non-nucleotide material.

The sense RNA strand may include a first 3′ overhang and/or the antisense RNA strand may include a second 3′ overhang in some embodiments, and in certain embodiments, the first and/or the second 3′ overhang comprises from 1 to about 6 nucleotides. In particular embodiments, the first and/or the second 3′ overhangs may be about 2 nucleotides, and in preferred embodiments, the first and/or the second 3′ overhangs may be a dinucleotide of dithymidine (TT) or diuridine (uu).

The siRNA of certain embodiments, may have a sense RNA strand having a sequence corresponding to a sequence selected from SEQ ID NO: 43, SEQ ID NO: 45, SEQ ID NO: 48, SEQ ID NO: 49, SEQ ID NO: 51, SEQ ID NO: 53, and SEQ ID NO: 56.

Other embodiments of the invention include a pharmaceutical composition including an effective amount of an isolated siRNA having a sense RNA strand including a nucleotide sequence substantially identical to a target sequence of about 19 to about 25 contiguous nucleotides a complement pathway protein mRNA or complement system regulatory protein mRNA and an antisense RNA strand having a sequence substantially complementary to the sense RNA strand wherein the sense RNA strand and the antisense RNA strand form an RNA duplex, and a pharmaceutically acceptable carrier. In various embodiments, the siRNA may include a sense RNA strand having a nucleotide sequence substantially identical to a target sequence of from about 19 to about 25 contiguous nucleotides of human classical, alternative, and lectin complement pathway proteins, C1, C2, C3, C4, C5, C6, C7, C8, C9, Factor B, Factor D, Factor H, Factor I, or vitronectin, heparin, clusterin, C4bBP, MCP and DAF, and a pharmaceutically acceptable carrier. In preferred embodiments, the siRNA may include a sense RNA strand having a nucleotide sequence substantially identical to a target sequence of from about 19 to about 25 contiguous nucleotides of human C3 protein mRNA, and a pharmaceutically acceptable carrier.

In some embodiments, the pharmaceutical composition may include a delivery reagent, and the delivery reagent may be lipofectin, lipofectamine, cellfectin, polycations, or liposomes, and combinations thereof. In particular embodiments, the delivery agent may be a liposome. The liposome of some embodiments may further include a targeting ligand that targets the liposome to cells at or near the site of a drusen, and in certain embodiments, the ligand may be an antibody. The liposome of other embodiments may be modified with an opsonization-inhibition moiety, and in particular embodiments, the opsonization-inhibiting moiety may include PEG, PPG, or derivatives thereof.

In various embodiments, the sense RNA strand may have a sequence corresponding to a sequence selected from SEQ ID NO: 43, SEQ ID NO: 45, SEQ ID NO: 48, SEQ ID NO: 49, SEQ ID NO: 51, SEQ ID NO: 53, and SEQ ID NO: 56, and in embodiments, the sense RNA strand may have a first 3′ overhang and/or the antisense RNA strand may have a second 3′ overhang. In some embodiments, the first and/or the second 3′ overhang may include from 1 to about 6 nucleotides, and in others, the first and/or the second 3′ overhang may be about 2 nucleotides. In particular embodiments, the first and/or the second 3′ overhangs may be a dinucleotide of dithymidine (TT) or diuridine (uu).

Still other embodiments of the invention include methods for inhibiting expression of human complement system or complement system regulator mRNA the methods including administering to a subject in need thereof an effective amount an isolated siRNA having a sense RNA strand including a nucleotide sequence substantially identical to a target sequence of about 19 to about 25 contiguous nucleotides of a complement pathway protein mRNA or complement system regulatory protein mRNA and an antisense RNA strand having a sequence substantially complementary to the sense RNA strand wherein the sense RNA strand and the antisense RNA strand form an RNA duplex, and degrading said human complement system or complement system regulatory mRNA wherein degrading inhibits expression of said human complement system or complement system regulatory mRNA. In various embodiments, the siRNA may include a sense RNA strand having a nucleotide sequence substantially identical to a target sequence of from about 19 to about 25 contiguous nucleotides of human classical, alternative, and lectin complement pathway proteins, C1, C2, C3, C4, C5, C6, C7, C8, C9, Factor B, Factor D, Factor H, Factor I, or vitronectin, clusterin, C4bBP, MCP and DAF. In preferred embodiments, the siRNA may include sense RNA strand having a nucleotide sequence substantially identical to a target sequence of from about 19 to about 25 contiguous nucleotides of human C3 protein mRNA, and human C3 protein RNA is degraded wherein degrading the human C3 protein mRNA inhibits expression of human C3 protein mRNA and inhibits initiation and/or proliferation of a complement system response.

In embodiments, the subject in need thereof may be a human being, and in certain embodiments, the effective amount of the siRNA may be from about 1 nM to about 100 nM.

In some embodiments, the siRNA may be expressed from a recombinant plasmid, and in others, the siRNA is expressed from a recombinant virus which may be an adenoviral vector, an adeno-associated viral vector, a lentiviral vector, a retroviral vector, or a herpes virus vector. In particular embodiments, the recombinant viral vector comprises an adeno-associated viral vector. In still other embodiments, the recombinant virus may express surface proteins from other viruses such as vesicular stomatitis virus, rabies virus, Ebola virus, or Mokola virus.

The isolated siRNA of embodiments may be administered enterally by methods including oral, rectal, and intranasal administration. In other embodiments, the siRNA may be administered parenterally, by methods including intravascular administration, peri- and intra-tissue injection, subcutaneous injection or deposition, or subcutaneous infusion administration, and direct administration at or near a site of drusen formation. In particular embodiments, intravascular administration may include intravenous bolus injection, intravenous infusion, intra-arterial bolus injection, intra-arterial infusion and catheter instillation into a vasculature, and in other embodiments, peri- and intra-tissue injection may include intra-retinal injection, subretinal injection and intra-vitreal. In certain embodiments, the siRNA may be administered at or near the site of drusen formation by a method including administration by catheter, retinal pellet, suppository, an implant comprising a porous material, an implant comprising a non-porous material, or an implant comprising a gelatinous material, and in particular embodiments, the siRNA may be administered by eye drops or intraorbital injection.

Further embodiments of the invention include methods of treating a disease in a subject, including administering to the subject an effective amount of an isolated siRNA having a sense RNA strand including a nucleotide sequence substantially identical to a target sequence of about 19 to about 25 contiguous nucleotides of a complement pathway protein mRNA or complement system regulatory protein mRNA and an antisense RNA strand having a sequence substantially complementary to the sense RNA strand wherein the sense RNA strand and the antisense RNA strand form an RNA duplex, and degrading said human complement system or complement system regulatory mRNA wherein degrading the inhibits expression of said human complement system or complement system regulatory mRNA. In various embodiments, the siRNA may include a sense RNA strand having a nucleotide sequence substantially identical to a target sequence of from about 19 to about 25 contiguous nucleotides of human classical, alternative, and lectin complement pathway proteins, C1, C2, C3, C4, C5, C6, C7, C8, C9, Factor B, Factor D, Factor H, Factor I, or vitronectin, heparin, clusterin, C4bBP, MCP and DAF. In preferred embodiments, the siRNA may include a sense RNA strand having a nucleotide sequence substantially identical to a target sequence of from about 19 to about 25 contiguous nucleotides of human C3 protein mRNA, and human C3 protein RNA is degraded wherein degrading the human C3 protein mRNA inhibits expression of human C3 protein mRNA and inhibits initiation and/or proliferation of a complement system response.

In various embodiments, the disease may be selected from macular degeneration-related disorder, age-related macular disorder, North Carolina macular dystrophy, Sorsby's fundus dystrophy, Stargardt's disease, pattern dystrophy, Best disease, dominant drusen, diabetic retinopathy and malattia leventinese, and macular degeneration-related disorder may include retinal detachment, chorioretinal degenerations, retinal degenerations, photoreceptor degenerations, RPE degenerations, mucopolysaccharidoses, rodcone dystrophies, cone-rod dystrophies, and cone degenerations. In particular embodiments, the disease may be age-related macular degeneration, or diabetic retinopathy.

In certain embodiments, the method of treating a disease may furthering include detecting the expression level of a human complement system mRNA or complement system regulatory mRNA with urine, blood plasma, serum, whole blood, or eye fluid from the subject.

Still further embodiments of the invention include methods of treating an ocular disorder associated with drusen including ocularly administering to at least one eye of a subject an effective amount of an isolated siRNA having a sense RNA strand including a nucleotide sequence substantially identical to a target sequence of about 19 to about 25 contiguous nucleotides of a complement pathway protein mRNA or complement system regulatory protein mRNA and an antisense RNA strand having a sequence substantially complementary to the sense RNA strand wherein the sense RNA strand and the antisense RNA strand form an RNA duplex, and degrading said human complement system or complement system regulatory mRNA wherein degrading the inhibits expression of said human complement system or complement system regulatory mRNA. In various embodiments, the siRNA may include a sense RNA strand having a nucleotide sequence substantially identical to a target sequence of from about 19 to about 25 contiguous nucleotides of human classical, alternative, and lectin complement pathway proteins, C1, C2, C3, C4, C5, C6, C7, C8, C9, Factor B, Factor D, Factor H, Factor I, or vitronectin, clusterin, C4bBP, MCP and DAF. In preferred embodiments, the siRNA may include a sense RNA strand having a nucleotide sequence substantially identical to a target sequence of from about 19 to about 25 contiguous nucleotides of human C3 protein mRNA, and human C3 protein RNA is degraded wherein degrading the human C3 protein mRNA inhibits expression of human C3 protein mRNA and inhibits initiation and/or proliferation of a complement system response

In various embodiments, the ocular disorder may be macular degeneration-related disorder, age-related macular disorder, North Carolina macular dystrophy, Sorsby's fundus dystrophy, Stargardt's disease, pattern dystrophy, Best disease, dominant drusen, diabetic retinopathy and malattia leventinese, and in particular embodiments, the disease may be age-related macular degeneration or diabetic retinopathy.

The ocular administration of embodiments may be topical, intra-retinal injection, subretinal injection, intravitreal injection, and intraorbital injection, and in some embodiments, the ocular administration is selected from eye drops or intraorbital injection.

In certain embodiments, the sense RNA strand may have a sequence corresponding to a sequence selected from SEQ ID NO: 43, SEQ ID NO: 45, SEQ ID NO: 48, SEQ ID NO: 49, SEQ ID NO: 51, SEQ ID NO: 53, and SEQ ID NO: 56, and in particular embodiments, the siRNA may have a first 3′ overhang and/or a second 3′ overhang selected from dithymidine (TT) or diuridine (uu).

### BRIEF DESCRIPTION OF DRAWINGS

For a fuller understanding of the nature and advantages of the present invention, reference should be had to the following detailed description taken in connection with the accompanying drawings, in which:

FIG. 1 illustrates human complement C3 expression in human cells treated with cytokines.

FIG. 2 illustrates a complement C3 siRNA screen in ARPE19 cells.

FIG. 3 illustrates the dose response of complement C3 siRNAs in ARPE19 cells.

FIG. 4 illustrates the cytotoxicity of C3 siRNAs in ARPE19 cells.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein are incorporated by reference in their entirety. Nothing herein is to be construed as an admission that the invention is not entitled to antedate such disclosure by virtue of prior invention.

It must also be noted that as used herein and in the appended claims, the singular forms “a”, “an”, and “the” include plural reference unless the context clearly dictates otherwise. Thus, for example, reference to an “eye” is a reference to one or more eyes and equivalents thereof known to those skilled in the art, and so forth.

As used herein, the term “about” means plus or minus 10% of the numerical value of the number with which it is being used. Therefore, about 50% means in the range of 45%-55%.

As used herein, a “subject” may include any human or non-human animal or mammal. Preferably, the subject is a human being.

“Administering” when used in conjunction with a therapeutic means to administer a therapeutic directly into or onto a target tissue or to administer a therapeutic to a patient whereby the therapeutic positively impacts the tissue to which it is targeted. Thus, as used herein, the term “administering”, when used in conjunction with complement regulatory agent, can include, but is not limited to, providing a complement regulatory agent into or onto the target tissue; providing a complement regulatory agent systemically to a patient by, e.g., intravenous injection whereby the therapeutic reaches the target tissue; providing a complement regulatory agent in the form of the encoding sequence thereof to the target tissue (e.g., by so-called gene-therapy techniques). “Administering” a composition may be accomplished by injection, topical administration, or by either method in combination with other known techniques.

The term “improves” is used to convey that the present invention changes either the appearance, form, characteristics and/or the physical attributes of the tissue to which it is being provided, applied or administered. The change in form may be demonstrated by any of the following alone or in combination: decrease in drusen size; improved vision; decreases RPE vascularization; improvement in wet and/or dry AMD related symptoms; delayed drusen formation; and delayed onset of wet and/or dry AMD.

As used herein, the term “therapeutic” means an agent utilized to treat, combat, ameliorate, prevent or improve an unwanted condition or disease of a patient. In part, embodiments of the present invention are directed to improve the functionality of the eye. As it applies to damage caused by drusen formation and AMD.

A “therapeutically effective amount” or “effective amount” of a composition is a predetermined amount calculated to achieve the desired effect, i.e., to effectively inhibit, prevent or decrease drusen formation, treat wet and/or dry AMD or its symptoms. A therapeutically effective amount of a complement regulatory agent of this invention is typically an amount such that when it is administered in a physiologically tolerable excipient composition, it is sufficient to achieve an effective local concentration in the tissue. Effective amounts of compounds of the present invention can be measured by improvements in sight, decrease in size and number of drusen, or delayed drusen formation and delayed onset of AMD or symptoms of AMD.

Generally speaking, the term “tissue” refers to any aggregation of similarly specialized cells which are united in the performance of a particular function. As used herein, “tissue”, unless otherwise indicated, refers to tissue which include, but are not limited to, the macula of the eye. For example, the macula comprises the choroid tissue, Bruch's membrane, retinal pigment epithelium (RPE), and photoreceptor cells (rod and cone cells).

As used herein, the term “agonist” is an agent that enhances or up regulates (e.g., potentiates or supplements) the production or activity of a gene product. An agonist can also be a compound which increases the interaction of a gene product, molecule or cell with another gene product, molecule or cell, for example, the agent may substitute a gene product with another homologous or heterologous gene product, or the agent may substitute a gene product with its receptor. In certain embodiments, an agonist may be a compound which enhances or increases binding or activation of a transcription factor to an upstream region of a gene thereby activating the gene. Any agent that activates gene expression, for example, by increasing RNA or protein synthesis or decreasing RNA or protein turnover, or activating a gene products activity may be an agonist whether the agent acts directly on the gene or gene product or acts indirectly, for example, by acting upstream in the gene regulation pathway. Examples of agonists of embodiments may be RNAs, peptides, antibodies and small molecules, or a combination thereof.

The term “antagonist”, as used here, is an agent that down regulates, suppresses, or inhibits the production or activity of a gene or gene product. For example, an antagonist can be an agent that inhibits or decreases the interaction between a gene product, molecule or cell and another gene product, molecule or cell. In certain embodiments, an antagonist may be a compound that inhibits or decreases binding or activation of a transcription factor to an upstream region of a gene thereby blocking activation of the gene. Any agent that inhibits gene expression or gene product activity may be an antagonist whether the agent acts directly on the gene or gene product or acts indirectly, for example, by acting upstream in the gene regulation pathway. An antagonist can also be a compound that down regulates expression of a gene or which reduces the amount of gene product present, for example, by decreasing RNA or protein synthesis or increasing RNA or protein turnover. In various embodiments, an antagonist may be RNAs, peptides, antibodies, and small molecules, or a combination thereof.

The term “antibody” or “immunoglobulin” is used to include intact antibodies and binding fragments thereof. Typically, fragments compete with the intact antibody from which they were derived for specific binding to an antigen fragment including separate heavy chains, light chains Fab, Fab′, F(ab′)2, Fabc, and Fv. Antibody fragments may be produced by any method known in the art, for example, by recombinant DNA techniques, or by enzymatic or chemical separation of intact immunoglobulins. The term “antibody” also includes one or more immunoglobulin chain that are chemically conjugated to, or expressed as, fusion proteins with other proteins. The term “antibody” also includes specific or bispecific antibody, and a specific or bifunctional antibody is an artificial hybrid antibody having two different heavy/light chain pairs and two different binding sites. Bispecific antibodies can be produced by a variety of methods including, but not limited to, fusion of hybridomas or linking of Fab fragments. See, e. g., Songsivilai & Lachmann, Clin. Exp. Immunol. 79: 315321 (1990); Kostelny et al., J. Immunol. 148, 1547-1553 (1992).

The term “antisense molecules” include antisense or sense oligonucleotides comprising a single-stranded nucleic acid sequence (either RNA or DNA) capable of binding to target mRNA (sense) or DNA (antisense) sequences for a specific protein (e. g., a complement pathway molecule). The ability to derive an antisense or a sense oligonucleotide, based upon a cDNA sequence encoding a given protein is described in, e.g., Stein and Cohen (Cancer Res. 48: 2659, 1988) and van der Krol et al. (BioTechniques 6: 958, 1988).

As used herein, a nucleic acid sequence “substantially identical” to a target sequence contained within the target mRNA is a nucleic acid sequence which is identical to the target sequence, or which differs from being identical to the target sequence by one or more nucleotides. Sense strands of the invention which comprise nucleic acid sequences substantially identical to a target sequence are characterized in that siRNA comprising such sense strands induce RNAi-mediated degradation of mRNA containing the target sequence. For example, an siRNA of the invention can comprise a sense strand comprise nucleic acid sequences which differ from a target sequence by one, two or three or more nucleotides, as long as RNAi-mediated degradation of the target mRNA is induced by the siRNA.

The complement system is constantly active at low levels in the normal eye and is closely regulated by intraocular complement regulatory proteins. Vitreous and aqueous humor from normal human eyes contain soluble forms of MCP, DAF and CD59. MCP is thought to interact with β1 integrin and may be involved in the adhesion of the retinal pigment epithelium (RPE) to its basement membrane and Bruch's membrane. Vitronectin is produced by photoreceptors, and the mean retinal RNA is nearly 50% of that measured in liver. C3 and C5 message has been identified in RPE, and C3, C5, and C9 transcripts have been detected in neural retina and/or choroid. Levels of C5 mRNA in cultured RPE cells is nearly equivalent to that found in liver-derived cells, and almost 60% of that of human liver, suggesting that the RPE is probably the main source of C5.

Complement components and regulators have also been localized to drusen. Drusen are focal deposits of extracellular material that are localized between the basal lamina of the RPE and the inner collagenous layer of Bruch's membrane. Their presence in the eye is a significant risk factor and indicator for both dry and wet forms of age-related macular degeneration (AMD). Drusen are composed of lipid, carbohydrate, protein and cellular components that are most likely of RPE origin. Immunohistochemical analysis of drusen revealed the presence of vitronectin, clusterin, IgG, MAC, C5, MCP and CR1. In addition, robust labeling was observed with antibodies raised against C3/C3d and the C3 activation fragments (C3b, iC3b, C3dg).

Similarly, tissue surrounding drusen also exhibited immunoreactivity for complement factors, C5, IgG, vitronectin, clusterin, components of MAC and MCP have been localized to RPE cells that are in close proximity of drusen. Furthermore, intense choroidal immunostaining was observed with antibodies against C3; however, little labeling was found in the rest of the choroid. Additionally, unknown structures that are generally deposited at sites of complement activation also showed strong immunoreactivity for proteolytic fragments of C3. It has been suggested that these structures are residual debris from degenerating RPE cells that have undergone cytolysis.

This hypothesis is further supported by animal studies. Mice deficient in monocyte chemoattractant protein-1 (Ccl-1, MCP-1) or its cognate C-C chemokine receptor-2 (Ccr-2) develop lipofusion in the RPE, drusen beneath the RPE, photoreceptor atrophy and choroidal neovascularization (CNV). In addition, complement and IgG deposits are found in the RPE and choroid, suggesting immunological components are responsible for the AMD phenotype.

The role of complement was examined in a mouse model of laser-induced CNV. Laser-photocoagulation in C57BL6 mice resulted in C3 and MAC deposition at the site of drusen formation. In contrast, laser photocoagulation did not induce CNV in C3 deficient mice or produce MAC deposition. Systemic administration of anti-C6 polyclonal antibodies significantly reduced the development of CNV and deposition of MAC at the site of the laser spots. Furthermore, when C57BL6 mice were systemically depleted of complement with cobra venom factor (CVF) and treated with laser, no C3 or MAC immunoreactivity was observed in the laser spots and the development of CNV was significantly reduced. CVF is a structural analog of C3 that functionally resembles C3b and binds Factor B to form CVFBb, a C3 and C5 convertase. CVF is resistant to inactivation by Factors H and I. Consequently, C3 and C5 are continuously hydrolyzed until the complement system is completely depleted. These findings suggest that activation of complement is necessary for the development of laser-induced CNV in mice and may also be necessary for the development of CNV in AMD.

Indeed, it has been confirmed that a mutation (Y402H) in Factor H is significantly associated with the development of AMD. This mutation is located in the heparin and C-reactive protein binding sites of Factor H, and the association is prominent in cases of both early AMD and CNV. This mutation may alter the behavior of the protein so that it can no longer regulate the complement pathway. For example, a change in its binding capabilities may prevent it from properly interacting with microbes or complement fragment C3b, and this dysfunction may eventually result in local tissue damage. In addition to the Y402H mutation, at least 7 other Factor H variants have been linked with AMD.

Staining of tissue from patients with AMD revealed intense and specific immunoreactivity for Factor H in drusen, in the subRPE space and around the choroidal capillaries and was most prominent in the macula. The distribution of Factor H in macula correlated with the distribution of MAC. In certain instances sub-structural elements within the drusen also reacted with antibodies directed against C3 fragments. Locations outside the macula had less Factor H and C5b-9 immunoreactivity, and very little or no C5b-9 immunoreactivity was observed in RPE or choroid from patients under the age of 50 and without AMD. Analysis of mRNA from freshly isolated RPE and the RPE/choroid complex, but not neural retina, derived from donor eyes with and without AMD, revealed that Factor H and its truncated isoform are abundantly expressed in RPE, with levels approaching that of liver.

The recent Factor H finding provides insights into the roles of some established risk factors for AMD. For example, it has been reported that there is a strong association between Chlamydia pneumoniae infection and AMD. This pathogen has been identified in neovascular tissue that was surgically removed from eyes with AMD and has a high affinity for vascular tissue, where it establishes infection. In a scenario where one does not have the ability to regulate complement, such a pathogen may stimulate a chronic inflammation that ultimately leads to tissue damage.

Another risk for AMD is smoking. Cigarette smoke can activate the alternative pathway by modifying C3 in vitro. Thus, in the absence of functional Factor H, cigarette smoke can lead to uncontrolled complement activation. Interestingly, there is another disease caused by a deficiency in Factor H that has an AMD-like phenotype. Membranoproliferative glomerulonephritis type II (MPGNII), which can also be caused by a point mutation in Factor H, results in severe glomerulonephritis and early onset drusen formation. The drusen are structurally and compositionally indistinguishable from those found in AMD.

Aspects of the present invention include compositions and methods for treating or preventing disease by regulating activation of the complement pathway. In some embodiments, the disease is an ocular disease, and in particular embodiments, the disease is macular degeneration related disorders including, for example, age-related macular disorder (AMD), North Carolina macular dystrophy, Sorsby's fundus dystrophy, Stargardt's disease, pattern dystrophy, Best disease, dominant drusen, diabetic retinopathy and malattia leventinese. Other diseases or disorders include retinal detachment, chorioretinal degenerations, retinal degenerations, photoreceptor degenerations, RPE degenerations, mucopolysaccharidoses, rod-cone dystrophies, cone-rod dystrophies, and cone degenerations.

One embodiment of the present invention provides methods for treating or preventing the development of a macular degeneration in a subject suffering from or at risk of developing a macular degeneration-related disorder. Such methods may include administering to the subject of an effective amount of a therapeutic agent which modulates an activity of at least one complement pathway associated molecule, or a cellular activity mediated by the complement pathway. In embodiments of the method, the subject may have a macular degeneration-related disorder, or the subject may be at risk of developing a macular degeneration-related disorder. In some embodiments, the subject may be free of complement-related diseases other than macular degeneration related disorders.

In embodiments of the methods, the therapeutic agent may modulate the activity of a complement pathway associated molecule by modulating the level of the complement pathway associated molecule or an up stream regulator of the complement pathway associated molecule, and in certain embodiments, the therapeutic agent may modulate the protein level of the complement pathway associated molecule. Non-limiting examples, of complement pathway associated molecule that may be modulated using methods of embodiments include anaphylatoxin C3a, anaphylatoxin C5a, C6, clusterin, haptoglobin, Ig kappa chain, Ig lambda chain, or Ig gamma chain. In other embodiments, the therapeutic agent may modulate an enzymatic activity of a complement protein or a complement pathway associated molecule. For example, the therapeutic agent may modulate the catalysis or conversion of C3 into C3a and C3b, conversion of C5 into C5a and C5b, or cleavage of Factor B into Ba and Bb.

In some embodiments, the method may further include detecting the level of the complement pathway associated molecule modulated using the therapeutic agent, and the level of complement pathway associated molecule may be determined before during or after treatment with the therapeutic agent. The level of complement pathway associated molecule may be detected in any way known in the art, for example, the level may be determined from bodily fluids such as urine, blood plasma, serum, whole blood, or eye fluid from the subject.

Therapeutic agents of various embodiments may include any substance, molecule, element, compound, entity, or a combination thereof, including, but not limited to, protein, peptide, oligopeptide, small organic molecule, polysaccharide, and polynucleotide such as siRNA and the like. The agent may be a natural product, synthetic compound, or a chemical compound, or a combination of two or more substances.

In preferred embodiments, isolated siRNA targeted to mRNA of complement proteins of the classical, alternative or Lectin pathways may be the therapeutic agent provided to a patient in need thereof. For example, siRNA of embodiments may be administered to a patient exhibiting symptoms of diseases involving improper complement activation and may be used to inhibit complement activation. In particular, complement proteins that may be targeted include, but are not limited to, classical, alternative, and lectin complement pathway proteins such as, for example, C1, C2, C3, C4, C5, C6, C7, C8, C9, Factor B, Factor D, Factor H, Factor I and proteins that modulate MAC formation on the cell surface such as vitronectin, heparin, clusterin, C4bBP, MCP and DAF are targeted. In preferred embodiments, C3, C5, and vitronectin may be targeted, preferably C3.

Embodiments of the invention are, therefore, directed to an isolated siRNA having a sense RNA strand including a sequence substantially identical a human complement system mRNA or complement system regulatory protein mRNA and an antisense RNA strand substantially complementary to the sense RNA strand. In preferred embodiments, the sense RNA strand may be substantially identical to human C3 mRNA.

Without wishing to be bound by theory, siRNA of the invention may cause the RNAi-mediated degradation of targeted mRNAs, thereby reducing or eliminating production of the targeted complement protein. For example, siRNA-mediated degradation of C3 or C5 mRNA may inhibit MAC formation and complement system activity because C3 is necessary for the formation of C3C5 convertase and the subsequent activation of C5 which is required for initiating MAC formation. Similarly, inactivation of vitronectin may prohibit the formation of MAC on the surface on the RPE preventing or reversing drusen formation.

As used herein, “target mRNA” means the messenger RNA that a specific siRNA molecule has been designed to eliminate. For example in embodiments of the invention, the target mRNA may be a human protein such as, for example, classical, alternative, and lectin complement pathway proteins, including, but not limited to, C1, C2, C3, C4, C5, C6, C7, C8, C9, Factor B, Factor D, Factor H, Factor I and proteins that modulate MAC formation on the cell surface such as vitronectin, clusterin, C4bBP, MCP and DAF, mutant or alternative splice forms of genes including but not limited to classical, alternative, and lectin complement pathway proteins, C1, C2, C3, C4, C5, C6, C7, C8, C9, Factor B, Factor D, Factor H, Factor I and proteins that modulate MAC formation on the cell surface such as vitronectin, clusterin, C4bBP, MCP and DAF.

As used herein, a gene or mRNA which is “cognate” to human gene that is a gene or mRNA from another mammalian species which is homologous the human gene. For example, the cognate C5 human mRNA may be the C5 mRNA from a mouse.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

In embodiments, the invention may provide for isolated siRNA comprising short double-stranded RNA from about 17 to about 29 nucleotides in length, or in some embodiments, from about 19 to about 25 nucleotides in length, that are targeted to a particular mRNA. In general, an siRNA may include a sense RNA strand and a substantially complementary antisense RNA strand annealed together by standard Watson-Crick base-pairing interactions (hereinafter “base-paired”). As described in more detail below, the sense strand may include a nucleic acid sequence that is identical or nearly identical to a sequence contained within the target mRNA.

Embodiments of the invention are, therefore, directed to an isolated siRNA having a sense RNA strand including a sequence substantially identical to 19 to 25 contiguous nucleotides of a human complement system mRNA or complement system regulatory protein mRNA and an antisense RNA strand substantially complementary to the sense RNA strand. In preferred embodiments, the sense RNA strand may be substantially identical to a 19 to 25 contiguous nucleotides of human C3 mRNA.

The sense and antisense strands of the present siRNA may consist of two separate complementary, single-stranded RNA molecules or include a single molecule in which two complementary portions are base-paired and are covalently linked by a single-stranded “hairpin” area. Without wishing to be bound by any theory, the hairpin of the latter siRNA molecule may be cleaved intracellularly by the “Dicer” protein (or its equivalent) to form an siRNA of two individual base-paired RNA molecules.

The siRNA of the invention can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to one or more end of the siRNA or to one or more internal nucleotides of the siRNA. Such alterations may include, but are not limited to, modifications that make the siRNA resistant to nuclease digestion.

One or both strands of the siRNA of the invention may also have a 3′ overhang. As used herein, a “3′ overhang” refers to at least one unpaired nucleotide, including ribonucleotides, deoxynucleotides, or combinations thereof extending from the 3′-end of an RNA strand. For example, in one embodiment, siRNA directed to complement mRNA may include at least one 3′ overhang of from 1 to about 6 nucleotides or from 1 to about 5 nucleotides in length. In some embodiments, the overhang may be from 1 to about 4 nucleotides in length, and in at least one embodiment, the overhang may be from about 2 to about 4 nucleotides in length. In still other embodiments, both strands of the siRNA molecule have a 3′ overhang. The length of which may be the same or different for each strand. In yet other embodiments, the 3′ overhang is present on both strands of the siRNA and may be 2 nucleotides in length. For example, each strand of the siRNA of the invention can comprise 3′ overhangs of dithymidine (“TT”) or diuridine (“uu”).

Embodiments of the invention are, therefore, directed to an isolated siRNA having a sense RNA strand including a sequence substantially identical to 19 to 25 contiguous nucleotides of a human complement system mRNA or complement system regulatory protein mRNA and an antisense RNA strand substantially complementary to the sense RNA strand wherein the sense RNA strand and/or the antisense RNA strand have 3′ overhangs. In preferred embodiments, the sense RNA strand may be substantially identical to a 19 to 25 contiguous nucleotides of human C3 mRNA and the sense RNA strand and/or the antisense RNA strand having 3′ overhangs. In particularly preferred embodiments, the siRNA may have a sense RNA strand substantially identical to human C3 mRNA and the sense RNA strand and/or the antisense RNA strand may have 3′ overhangs of 2 nucleotides with the nucleotides each being deoxythymidine or deoxyuridine.

In order to enhance the stability of the present siRNA, the 3′ overhangs can be stabilized against degradation. In one embodiment, the overhangs are stabilized by including purine nucleotides, such as adenosine or guanosine nucleotides. Alternatively, substitution of pyrimidine nucleotides by modified analogues, for example, substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

In certain embodiments, the siRNA of the invention may include the sequence AA(N19)TT or NA(N21), where N is any nucleotide. These siRNA may include approximately 30-70% G/C, and preferably comprise approximately 50% G/C. The sequence of the sense siRNA strand corresponds to (N19)TT or N21 (i.e., positions 3 to 23), respectively. In the latter case, the 3′ end of the sense siRNA is converted to TT. The rationale for this sequence conversion is to generate a symmetric duplex with respect to the sequence composition of the sense and antisense strand 3′ overhangs. The antisense RNA strand is then synthesized as the complement to positions 1 to 21 of the sense strand.

Because position 1 of the 23-nt sense strand in these embodiments may not be recognized in a sequence-specific manner by the antisense strand, the 3′-most nucleotide residue of the antisense strand can be chosen deliberately. However, the penultimate nucleotide of the antisense strand (complementary to position 2 of the 23-nt sense strand in either embodiment) may generally be complementary to the targeted sequence.

In another embodiment, the siRNA of the invention may include the sequence NAR(N17)YNN, where R is a purine (e.g., A or G) and Y is a pyrimidine (e.g., C or U/T). The respective 21-nt sense and antisense RNA strands of this embodiment, therefore, may generally begin with a purine nucleotide. Such siRNA can be expressed from pol III expression vectors without a change in targeting site, as expression of RNAs from pol III promoters is only believed to be efficient when the first transcribed nucleotide is a purine.

The siRNA of the invention can be targeted to any stretch of approximately 19-25 contiguous nucleotides in any of the target mRNA sequences (the “target sequence”). Techniques for selecting target sequences for siRNA are given, for example, in Tuschl T et al., “The siRNA User Guide,” revised May 6, 2004, the entire disclosure of which is herein incorporated by reference. “The siRNA User Guide” is available on the world wide web at a website maintained by Dr. Thomas Tuschl, Department of Cellular Biochemistry, AG 105, Max-Planck-Institute for Biophysical Chemistry, 37077 Gottingen, Germany, and can be found by accessing the website of the Max Planck Institute and searching with the keyword “siRNA.” Thus, the sense strand of the present siRNA comprises a nucleotide sequence identical to any contiguous stretch of about 19 to about 25 nucleotides in the target mRNA.

Generally, a target sequence on the target mRNA can be selected from a given cDNA sequence corresponding to the target mRNA, preferably beginning 50 to 100 nt downstream (i.e., in the 3′ direction) from the start codon. The target sequence can, however, be located in the 5′ or 3′ untranslated regions, or in the region nearby the start codon. For Example, target sequences SEQ ID NOS. 1 to 26 and 57 in Table 1, which are within 100 nt of the 5′-end of the human C3 cDNA SEQ ID NO. 4 and correspond with target sequences SEQ ID NO. 27 to 41 and corresponding siRNA sequences SEQ ID NOS. 42 to 56.

TABLE 1

| ID NO | Source |
|---|---|
| 1 | Human C1 r |
| 2 | Human C1 s |
| 3 | Human C2 |
| 4 | Human C3 |
| 5 | Human C4a |
| 6 | Human C4b |
| 7 | Human C5 |
| 8 | Human C6 |
| 9 | Human C7 |
| 10 | Human C8 (alpha) |
| 11 | Human C8 (beta) |
| 12 | Human C8 (gamma) |
| 13 | Human C9 |
| 14 | Human Factor B |
| 15 | Human Factor D |
| 16 | Human Factor H-2 |
| 17 | Human Factor H-4 |
| 18 | Human Factor H˜5 |
| 19 | Human Factor I |
| 20 | Human Vitronectin |
| 21 | Human Clusterin (Retinal) |
| 22 | Human Clusterin-1 |
| 23 | Human Clusterin-2 |
| 24 | Human C4bBP (alpha) |
| 25 | Human MCP |
| 26 | Human DAF |
| 27 | C3 Target 1 |
| 28 | C3 Target 2 |
| 29 | C3 Target 3 |
| 30 | C3 Target 4 |
| 31 | C3 Target 5 |
| 32 | C3 Target 6 |
| 33 | C3 Target 7 |
| 34 | C3 Target 8 |
| 35 | C3 Target 9 |
| 36 | C3 Target 10 |
| 37 | C3 Target 11 |
| 38 | C3 Target 12 |
| 39 | C3 Target 13 |
| 40 | C3 Target 14 |
| 41 | C3 Target 15 |
| 42 | C3 siRNA 1 |
| 43 | C3 siRNA 2 |
| 44 | C3 siRNA 3 |
| 45 | C3 siRNA 4 |
| 46 | C3 siRNA 5 |
| 47 | C3 siRNA 6 |
| 48 | C3 siRNA 7 |
| 49 | C3 siRNA 8 |
| 50 | C3 siRNA 9 |
| 51 | C3 siRNA 10 |
| 52 | C3 siRNA 11 |
| 53 | C3 siRNA 12 |
| 54 | C3 siRNA 13 |
| 55 | C3 siRNA 14 |
| 56 | C3 siRNA 15 |
| 57 | Human C4bBP (beta) |

Splice variants of human complement system proteins have been identified for factors C2 and MCP, and an exonic splicing enhancer has been identified. siRNA directed to these and other as yet unidentified splice variants to complement system proteins are encompassed by the present invention.

Alternatively spliced variants of human complement system proteins may be identified as known in the art, for example, by using a technique called “RNAse protection”. RNAse protection involves transcription of a gene sequence into synthetic RNA, which is hybridized to RNA derived from other cells; for example, cells from tissue at or near the site of complement activation. The hybridized RNA is then incubated with enzymes that recognize RNA:RNA hybrid mismatches. Smaller than expected fragments indicate the presence of alternatively spliced mRNAs. In embodiments, putative alternatively spliced mRNAs may be cloned and sequenced by methods well known to those skilled in the art and used to develop siRNA.

Alternatively spliced complement pathway proteins may be identified using RT-PCR. In RT-PCR, mRNA from the diseased tissue is converted into cDNA using methods well-known to those of ordinary skill in the art, for example, by using the enzyme reverse transcriptase. The entire coding sequence of the cDNA may then be amplified via PCR using a forward primer located in the 3′ untranslated region, and a reverse primer located in the 5′ untranslated region. The amplified products can be analyzed for alternative splice forms, for example by comparing the size of the amplified products with the size of the expected product from normally spliced mRNA, for example, by agarose gel electrophoresis. Any change in the size of the amplified product can indicate alternative splicing. Alternatively spliced variants may then be sequenced and used to develop siRNA.

mRNA produced from mutant genes can also be identified through the techniques described above for identifying alternative splice forms. As used herein, “mutant” complement pathway proteins are produced from a gene having a sequence that differs in sequence from the complement pathway protein sequences set forth herein for example in SEQ ID NOS 1-26 and 57 at one or more base pair. Thus, allelic forms of these genes, and the mRNA produced from them, are considered “mutants” for purposes of this invention.

Databases containing nucleotide sequences related to a given disease gene can be used to identify alternatively spliced or mutant mRNAs. For example, useful databases may include GenBank, Embase, and the Cancer Genome Anatomy Project (CGAP) database. The CGAP database, for example, contains expressed sequence tags (ESTs) from various types of human cancers. An mRNA or gene sequence from including but not limited to classical, alternative, and lectin complement pathway proteins, C1, C2, C3, C4, C5, C6, C7, C8, C9, Factor B, Factor D, Factor H, Factor I and proteins that modulate MAC formation on the cell surface such as vitronectin, clusterin, C4bBP, MCP and DAF genes can be used to query such a database to determine whether ESTs representing alternatively spliced mRNAs have been found for a these genes.

The siRNA of the invention can be obtained using a number of techniques known to those of skill in the art, including, but not limited to, chemical synthesis or recombinantly produced using methods. For example, siRNA may be produced using the Drosophila in vitro system described in U.S. published application 2002/0086356 of Tuschl et al., the entire disclosure of which is herein incorporated by reference or siRNA may be chemically synthesized using appropriately protected ribonucleoside phosphoramidites and a conventional DNA/RNA synthesizer. In embodiments, siRNA may be synthesized as two separate, complementary RNA molecules that are combined following synthesis, or a single RNA molecule with two complementary regions may be synthesized that is capable of binding to itself to form a “hairpin” structure. Synthetic RNA molecules or synthesis reagents commercially available are also encompassed by embodiments of the invention.

Alternatively, siRNA can be expressed from recombinant circular or linear DNA plasmids using a suitable promoter. Suitable promoters for expressing siRNA of the invention from a plasmid are well known in the art and may include, for example, the U6 or H1 RNA pol III promoter sequences and the cytomegalovirus promoter. Selection of other suitable promoters is within the skill in the art. The recombinant plasmids of the invention may also include inducible or regulatable promoters for expression of the siRNA in a particular tissue or in a particular intracellular environment.

siRNA expressed from recombinant plasmids may be isolated from cultured cell expression systems using standard techniques or expressed intracellularly in the diseased tissue, for example, in or near the eye, in or near the ocular cavity, or at or near area of drusen formation in vivo. The use of recombinant plasmids to deliver siRNA of the invention to cells in vivo is discussed in more detail below. In various embodiments, a single siRNA can be expressed from a recombinant plasmid as either as two separate, complementary RNA molecules, or as a single RNA molecule with two complementary regions.

Selection of plasmids suitable for expressing siRNA of the invention, methods for inserting nucleic acid sequences for expressing the siRNA into the plasmid and methods of delivering the recombinant plasmid to the cells of interest are vary in embodiments and may be selected from any plasmid known in the art or subsequently developed. For example, See Tuschl, T. (2002), Nat. Biotechnol, 20: 446-448; Brummelkamp T R et al. (2002), Science 296: 550-553; Miyagishi M et al. (2002), Nat. Biotechnol. 20: 497-500; Paddison P J et al. (2002), Genes Dev. 16: 948-958; Lee N S et al. (2002). Nat. Biotechnol. 20: 500-505; and Paul C P et al. (2002), Nat. Biotechnol. 20: 505-508, the entire disclosures of which are herein incorporated by reference.

As used herein, “in operable connection with a polyT termination sequence” means that the nucleic acid sequences encoding the sense or antisense strands are immediately adjacent to the polyT termination signal in the 5′ direction. During transcription of the sense or antisense sequences from the plasmid, the polyT termination signals act to terminate transcription.

As used herein, “under the control” of a promoter means that the nucleic acid sequences encoding the sense or antisense strands are located 3′ of the promoter, so that the promoter can initiate transcription of the sense or antisense coding sequences.

Recombinant viral vectors of embodiments may include sequences encoding the siRNA of the invention, any suitable promoter for expressing the siRNA sequences, and termination signals known in the art, for example, a polyT termination signal. Selection of other suitable promoters is within the skill in the art, and suitable promoters may include, for example, the U6 or H1 RNA pol III promoter sequences or the cytomegalovirus promoter. In some embodiments, recombinant viral vectors may include inducible or regulatable promoters for expression of the siRNA in a particular tissue, in a particular intracellular environment, or in response to a particular intracellular or extracellular signal. siRNA of the invention can be expressed from a recombinant viral vector either as two separate, complementary RNA molecules, or as a single RNA molecule with two complementary regions.

Selection of recombinant viral vectors suitable for use in the invention, methods for inserting nucleic sequences for expressing the siRNA into the vector, and methods of delivering the viral vector to the cells of interest are within the skill in the art. See, for example, Domburg R (1995), Gene Therap. 2: 301-310; Eglitis M A (1998), Biotechniques 6: 608-614; Miller A D (1990), Hum Gene Therap. 1: 5-14; and Anderson W F (1998), Nature 392: 25-30, the entire disclosures of which are herein incorporated by reference.

Any viral vector capable of accepting the coding sequences for the siRNA molecule(s) to be expressed can be used, for example vectors derived from adenovirus (AV); adeno-associated virus (AAV); retroviruses (e.g., lentiviruses (LV), Rhabdoviruses, murine leukemia virus); herpes virus, and the like. The tropism of the viral vectors can also be modified by pseudotyping the vectors with envelope proteins or other surface antigens from other viruses. For example, an AAV vector of the invention can be pseudotyped with surface proteins from vesicular stomatitis virus (VSV), rabies, Ebola, Mokola, and the like. In various embodiments, viral vectors are those derived from AV and AAV. In a particular embodiment, the siRNA of the invention is expressed as two separate, complementary single-stranded RNA molecules from a recombinant AAV vector comprising, for example, either the U6 or H1 RNA promoters, or the cytomegalovirus (CMV) promoter.

Methods for expressing siRNA, constructing recombinant viral vectors, and delivering viral vectors into target cells are well known in the art. For example, a suitable AV vector for expressing the siRNA, a method for constructing the recombinant AV vector, and a method for delivering the vector into target cells are described in Xia H et al. (2002), Nat. Biotech. 20: 1006-1010 the entire disclosure of which is herein incorporated by reference, and suitable AAV vectors for expressing the siRNA of the invention, methods for constructing the recombinant AV vector, and methods for delivering the vectors into target cells are described in Samulski R et al. (1987), J. Virol. 61: 3096-3101; Fisher K J et al. (1996), J. Virol., 70: 520-532; Samulski R et al. (1989), J. Virol. 63: 3822-3826: U.S. Pat. No. 5,252,479; U.S. Pat. No. 5,139,941; International Patent Application No. WO 94/13788; and International Patent Application No. WO 93/24641, the entire disclosures of which are herein incorporated by reference.

In some embodiments, the ability of an siRNA containing a given target sequence to cause RNAi-mediated degradation of the target mRNA may be evaluated using standard techniques for measuring the levels of RNA or protein in cells. For example, siRNA of the invention can be delivered to cultured cells, and the levels of target mRNA can be measured by Northern blot or dot blotting techniques, or by quantitative RT-PCR. Alternatively, the levels of complement pathway proteins or proteins that modulate in the cultured cells can be measured by ELISA or Western blot.

In other embodiments, RNAi-mediated degradation of target mRNA by an siRNA containing a given target sequence can also be evaluated with animal models. For example, the effectiveness of a specific siRNA for treating AMD may be evaluated using CNV mouse models, and areas of macular injury in a CNV mouse can be measured before and after administration of a siRNA. A reduction in the complement activation in these models upon administration of the siRNA indicates the down-regulation of the target mRNA or improvement in the macular injury.

As discussed above, the siRNA of the invention target and cause RNAi-mediated degradation of complement pathway mRNA or modulators of complement system activation mRNA, or alternative splice forms, mutants or cognates thereof. Degradation of the target mRNA by siRNA of the invention may reduce the production of a functional gene product from complement pathway proteins, or proteins that modulate complement system activation on the cell surface. Thus, the invention provides a method for inhibiting expression of complement pathway proteins, or proteins that modulate complement system activation on the cell surface in a subject, wherein the siRNA of the invention is administered in an effective amount to a subject such that the target mRNA is degraded. As the products of the complement pathway, or modulators of the complement system genes are required for initiating complement system activation, the invention also provides a method of inhibiting complement system activation in a subject by the RNAi-mediated degradation of the target mRNA using siRNA of the invention.

Inhibition of complement system activation can be evaluated, for example, by directly measuring the progress of AMD related drusen formation in a subject, for example, drusen can be observed by ophthalmoscopy or by observing the number of complement proteins in the drusen before and after treatment with the siRNA of the invention. An inhibition or drusen formation is indicated if the number complement proteins in the drusen stays the same, is reduced, or is retarded. Techniques for observing and measuring the number complement proteins in tissue samples from a subject are within the skill in the art as discussed above, for example, RNAi-mediated degradation of target mRNA can be detected by measuring levels of the target mRNA or protein in the cells of a subject using standard techniques for isolating and quantifying mRNA or protein as described above. Inhibition of complement system activation can also be inferred through observing a change or reversal in a pathogenic condition associated with uncontrolled complement system activation. For example, in AMD, a slowing, halting or reversal of vision loss indicates an inhibition of drusen formation in the choroid.

It is understood that the siRNA can degrade the target mRNA (and thus inhibit complement system activation) in substoichiometric amounts. Without wishing to be bound by any theory, it is believed that the siRNA of the invention causes degradation of the target mRNA in a catalytic manner. Thus, compared to standard anti-complement system therapies, significantly less siRNA needs to be delivered at or near the site of uncontrolled complement system activation to have a therapeutic effect.

In embodiments, isolated siRNA is administered to a subject in an effective amount. One skilled in the art can readily determine an effective amount of the siRNA of the invention to be administered to a given subject, by taking into account factors such as the size and weight of the subject; the extent of the complement system activation or disease penetration; the age, health and sex of the subject; the route of administration; and whether the administration is regional or systemic. Generally, an effective amount of the siRNA of the invention comprises an intercellular concentration at or near of the site uncontrolled complement system activation of from about 1 nanomolar (nM) to about 100 nM, from about 2 nM to about 50 nM, or from about 2.5 nM to about 10 nM. In particular embodiments, from about 0.01 mg to about 10 mg of an siRNA may be an effective amount. However, in certain embodiments, greater or lesser amounts of siRNA may be administered.

In some embodiments, methods of the invention may be used to inhibit complement system activation formation which is non-pathogenic such as complement system activation which results from normal processes in the subject. Examples of non-pathogenic complement system activation include non-antigen specific immune response against bacteria and viruses. Thus, the invention provides a method for inhibiting non-pathogenic complement system activation for controlling immune response in relation to organ transplantation, xenotransplantation, and MI. In other embodiments, methods of the invention may be used to inhibit complement activation associated with a disease with pathogenicity associated with inappropriate or uncontrolled complement system activation such as, for example, uncontrolled complement system activation linked to autoimmune diseases. Other uncontrolled complement system diseases include diabetic retinopathy, age-related macular degeneration (AMD), systemic lupus erythematosus (SLE), sepsis, immune complex disease, inflammation, pulmonary and hepatic fibrosis, asthma, atherosclerosis, diabetes and Alzheimer's disease. These diseases are generally characterized by the destruction of normal tissue by uncontrolled complement activation. For example, in AMD, the choroid is invaded and destroyed by drusen in the RPE inhibiting the sight of the patient, and drusen-driven destruction of the choroid in AMD eventually leads to partial or full blindness. Therefore, in certain embodiments, siRNA may be used to inhibit drusen formation in AMD.

For treating complement system related diseases, siRNA of the invention can be administered to a subject in combination with a pharmaceutical agent which is different from the present siRNA. Alternatively, the siRNA of the invention can be administered to a subject in combination with another therapeutic method designed to treat the complement system related disease. For example, the siRNA of the invention can be administered in combination with therapeutic methods currently employed for treating autoimmune diseases (e.g., C1 INH).

In embodiments, siRNA of the invention can be administered to the subject either as naked siRNA, in conjunction with a delivery reagent, or as a recombinant plasmid or viral vector which expresses the siRNA. Suitable delivery reagents for administration in conjunction with the present siRNA include the Mirus Transit TKO lipophilic reagent; lipofectin; lipofectamine; cellfectin; or polycations (e.g., polylysine), or liposomes. In particular embodiments, the delivery reagent is a liposome.

Liposomes can aid in the delivery of the siRNA to a particular tissue, such as retinal or diseased tissue and can increase the blood half-life of the siRNA. Liposomes suitable for use in the invention may be formed using standard vesicle-forming lipids, which may include neutral or negatively charged phospholipids and a sterol, such as cholesterol. The selection of lipids may generally be guided by consideration of factors such as the desired liposome size and half-life of the liposomes in the blood stream. A variety of methods are known for preparing liposomes, for example as described in Szoka et al. (1980), Ann. Rev. Biophys. Bioeng. 9: 467; and U.S. Pat. Nos. 4,235,871, 4,501,728, 4,837,028, and 5,019,369, the entire disclosures of which are herein incorporated by reference.

In some embodiments, liposomes encapsulating the siRNA of the invention may further include a ligand molecule that can target the liposome to a particular cell or tissue at or near the site of complement system activation. Ligands may include proteins that bind to receptors prevalent in tumor or vascular endothelial cells such as, for example, antibodies that bind to tumor antigens or endothelial cell surface antigens.

In other embodiments, liposomes encapsulating siRNA of the invention may be modified to avoid clearance by the mononuclear macrophage and reticuloendothelial systems. For example, in embodiments, the liposome may include opsonization-inhibition moieties bound attached to the surface of the liposome. Opsonization-inhibiting moieties for use in preparing the liposomes are typically large hydrophilic polymers that are bound to the liposome membrane. As used herein, an opsonization inhibiting moiety is “bound” to a liposome membrane when it is chemically or physically attached to the membrane, for example, by the intercalation of a lipid-soluble anchor into the membrane itself, or by binding directly to active groups of membrane lipids. These opsonization-inhibiting hydrophilic polymers form a protective surface layer which significantly decreases the uptake of the liposomes by the macrophage-monocyte system (“MMS”) and reticuloendothelial system (“RES”) as described in U.S. Pat. No. 4,920,016, the entire disclosure of which is herein incorporated by reference. Opsonization inhibiting moieties suitable for modifying liposomes are preferably water-soluble polymers with a molecular weight from about 500 to about 40,000 daltons, and more preferably from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM₁. Copolymers of PEG, methoxy PEG, or methoxy PPG, or derivatives thereof, are also suitable. In addition, the oposonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; laminated polysaccharides or oligosaccharides (linear or branched); or carboxylated polysaccharides or oligosaccharides, e.g., reacted with derivatives of carbonic acids with resultant linking of carboxylic groups. In particular embodiments, the oposonization-inhibiting moiety is a PEG, PPG, or derivatives thereof. Liposomes modified with PEG or PEG-derivatives are sometimes called “PEGylated liposomes.”

The opsonization inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. Similarly, a dextran polymer can be derivatized with a stearylamine lipid-soluble anchor via reductive amination using Na(CN)BH₃ and a solvent mixture such as tetrahydrofuran and water in a 30:12 ratio at 60° C.

Liposomes modified with opsonization-inhibition moieties remain in the circulation much longer than unmodified liposomes. For this reason, such liposomes are sometimes called “stealth” liposomes. Stealth liposomes are known to accumulate in tissues fed by porous or “leaky” microvasculature. Thus, target tissue characterized by such microvasculature defects, for example solid tumors, will efficiently accumulate these liposomes; see Gabizon, et al. (1988) P.N.A.S., USA, 18: 6949-53. In addition, the reduced uptake by the RES lowers the toxicity of stealth liposomes by preventing significant accumulation in the liver and spleen. Thus, liposomes of the invention that are modified with opsonization-inhibition moieties can deliver the present siRNA to tumor cells.

In one embodiment, a liposome of the invention can comprise both opsonization-inhibition moieties and a ligand.

In still other embodiments, the siRNA of the invention can be administered to the subject by any means suitable for mechanically delivering the siRNA to the cells of the tissue at or near the area of uncontrolled complement system activation. For example, the siRNA can be administered by gene gun, or electroporation.

siRNA and pharmaceutical compositions of the invention may be delivered by any route known in the art. For example, suitable enteral administration routes may include eye drops, oral, rectal, or intranasal delivery. Suitable parenteral administration routes may include intravascular administration (e.g., intravenous bolus injection, intravenous infusion, intra-arterial bolus injection, intra-arterial infusion and catheter instillation into the vasculature); peri- and intra-tissue injection (e.g., intra-retinal injection, subretinal or intra-vitreal injection); subcutaneous injection or deposition including subcutaneous infusion (such as by osmotic pumps); direct application to the area at or near the site of uncontrolled complement system activation, for example by a catheter or other placement device (e.g., a retinal pellet or a suppository or an implant comprising a porous, non-porous, or gelatinous material); and inhalation. In certain embodiments, injections or infusions of siRNA of the invention may be given at or near the site of complement system activation.

In various embodiments, siRNA of the invention can be administered in a single dose or in multiple doses. One skilled in the art can also readily determine an appropriate dosage regimen for administering the siRNA of the invention to a given subject. For example, the siRNA can be administered to the subject once, for example as a single injection or deposition at or near the site of uncontrolled complement system activation. Alternatively, the siRNA can be administered once or twice daily to a subject for a period of from about three to about twenty-eight days, or from about seven to about ten days. For example, in some embodiments, the siRNA may be injected at or near the site of uncontrolled complement system activation once a day for seven days. Where a dosage regimen comprises multiple administrations, it is understood that the effective amount of siRNA administered to the subject can comprise the total amount of siRNA administered over the entire dosage regimen.

The siRNA of the invention are preferably formulated as pharmaceutical compositions prior to administering to a subject, according to techniques known in the art. Pharmaceutical compositions of the present invention are characterized as being at least sterile and pyrogen-free. As used herein, “pharmaceutical formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is herein incorporated by reference.

The present pharmaceutical formulations comprise an siRNA of the invention (e.g., 0.1 to 90% by weight), or a physiologically acceptable salt thereof, mixed with a physiologically acceptable carrier medium such as, for example, water, buffered water, normal saline, 0.4% saline, 0.3% glycine, hyaluronic acid and the like.

Pharmaceutical compositions of the invention can also include conventional pharmaceutical excipients and/or additives. Suitable pharmaceutical excipients include stabilizers, antioxidants, osmolality adjusting agents, buffers, and pH adjusting agents. Suitable additives include physiologically biocompatible buffers (e.g., tromethamine hydrochloride), additions of chelants (such as, for example, DTPA or DTPA-bisamide) or calcium chelate complexes (as for example calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized.

For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like. For example, a solid pharmaceutical composition for oral administration can comprise any of the carriers and excipients listed above and for example about 10-95%, in a further example 25%-75%, of one or more siRNA of the invention. A pharmaceutical composition for aerosol (inhalational) administration can comprise 0.01-20% by weight, in a further example 1%-10% by weight, of one or more siRNA of the invention encapsulated in a liposome as described above, and propellant. A carrier can also be included as desired; e.g., lecithin for intranasal delivery.

The invention will now be illustrated with the following non-limiting examples.

### EXAMPLES

### Materials and Methods

Cell Culture

293 cells (human embryonic kidney cells), ARPE19 cells (human retinal epithelial cells), and A549 cells (human lung carcinoma cells) were obtained from ATCC (Manassas, Va.) and cultured in Dulbecco's modified eagle medium (DMEM, Gibco, Carlsbad, Calif.) DMEM/F12 medium (Gibco) or F12-K medium (ATCC) respectively, with 10% fetal bovine serum (FBS; JRH Biosciences, Lenexa, Kans.) and an antibiotic-antimycotic reagent (Gibco).

siRNAs

Fifteen siRNAs targeting human complement C3 were synthesized by Integrated DNA Technologies (Coralville, Iowa). The siRNA target sequences are listed in Table 2.

TABLE 2
C3 sIRNA Targeting Sequences

| | |
|---|---|
| siRNA Name | Targeting Sequence 5′-3′ |

| | |
|---|---|
| SEQ. ID. NO. 27 | AAT CCG AGC CGT TCT CTA CAA |
| SEQ. ID. NO. 28 | AAC AAG CTC TGC CGT GAT GAA |
| SEQ. ID. NO. 29 | AAT GGA CAA AGT CGG CAA GTA |
| SEQ. ID. NO. 30 | AAC TAC ATG AAC CTA CAG AGA |
| SEQ. ID. NO. 31 | AAA AAG CGG CCA GTC AGA AGA |
| SEQ. ID. NO. 32 | AAT GAT TGG TGG ATT ACG GAA |
| SEQ. ID. NO. 33 | AAG TCC TCG TTG TCC GTT CCA |
| SEQ. ID. NO. 34 | AAA TGA TTG GTG GAT TAC GGA |
| SEQ. lD. NO. 35 | AAT TAC CGG CAG AAC CAA GAG |
| SEQ. ID. NO. 36 | AAA TGG AAT CTC TAC GAA GCT |
| SEQ. ID. NO. 37 | AAT GAA CAG AGA TAC TAC GGT |
| SEQ. ID. NO. 38 | AAG CCT TGG CTC AAT ACC AAA |
| SEQ. ID. NO. 39 | AAG CGC ATT CCG ATT GAG GAT |
| SEQ. ID. NO. 40 | AAT GGA ATC TCT ACG AAG CTC |
| SEQ. ID. NO. 41 | AAC CTC ATC GCC ATC GAC TCC |

An additional siRNA, targeting enhanced green fluorescent protein (EGFP), served as a negative control (Dharmacon, Lafayette, Colo.). The sense strand sequence of the siRNA was 5′-GGC UAC GUC CAG GAG CGC AdTdT 3′ and the antisense strand sequence was 5′ U GCG CUC CUG GAC GUA GCCdTdT-3′.

Cytokine Treatments and Western Blot Analysis

293 cells, ARPE19 and A549 cells were cultured in 24 well plates at 37° C. with 5% CO₂ overnight. The next day, cells were treated with the following cytokines (R & D systems, Minneapolis, Minn.): interleukin 1β (IL-1β; 100 ng/mL), interleukin 6 (IL-6; 250 ng/mL) and interferon γ (IFN-γ; 500 ng/mL).

Two days post-treatment, cell supernatants were collected from each well, and a human complement C3 Western blot was performed. Briefly, supernatants (25 μLs) were electrophoresed on a NuPAGE Novex 4-12% Bis-Tris gel (Invitrogen, Carlsbad, Calif.). The protein was transferred to nitrocellulose membrane and C3 was detected with a chicken anti-human complement C3 primary antibody (100 ng/mL) (Chemicon, Temecula, Calif.), an alkaline phosphatase conjugated rabbit anti-chicken IgY secondary antibody (1:10,000, Chemicon) and alkaline phosphatase detection reagent (Novagen, Madison, Wis.).

Human Complement siRNA Screening and Dose Response Assay

ARPE19 cells were cultured in 24 well plates at 37° C. with 5% CO₂ overnight. The next day, transfections were performed when cells were about 70% confluent. Cells were transfected with 5 nM, 25 nM, 125 nM or 625 nM siRNA mixed in a calcium phosphate (CaPi) reagent. Controls included transfection reagent lacking siRNA and nonspecific siRNA (EGFP1 siRNA). Briefly, siRNA was added to 20 μL of 250 mM CaCl₂ solution. The siRNA/CaCl₂ mixture was added drop-wise to 20 μL of 2× Hanks Balanced Salt Solution (HBS), while mixing by vortex. The siRNA/CaCl₂/HBS complex was added directly to the medium (300 μL/well). After 4 hours of incubation at 37° C., the medium was removed, and the cells were further incubated with 10% DMSO-containing serum-free medium (300 μL/well) at room temperature for 1-2 minutes. This medium was then removed, and the cells were fed again with 200 μL/well growth medium.

Complement C3 stimulation was performed four hours after transfection with IL-1β at a final concentration of 100 ng/mL. 48 hours post-transfection, the supernatant was collected from each well, and samples were analyzed via Western blotting.

Cytotoxicity Assay

After the supernatant was removed from cells, a cytotoxicity assay was performed. Complete growth medium containing 10% alamarBlue™ (Biosource, Camarillo, Calif.) was added to each well, and the cells were incubated at 37° C. with 5% CO₂ for 3 hours. Cytotoxicity was determined spectrophotometrically at 570 nm using an AD340 plate reader (Beckman Coulter). The alamarBlue™ Assay incorporates an oxidation-reduction (REDOX) indicator that responds to the chemical reduction of growth medium resulting from cell growth. In the presence of growing cells it changes from the oxidized (non-fluorescent, blue color) form to the reduced (fluorescent, red color) form.

### Example 1

Cytokines Upregulate C3 Expression in Cells

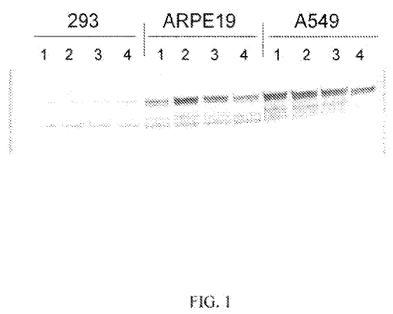

Human complement C3 expression was tested in cells following 2 days of cytokine treatment (Refer to FIG. 1 for Example 1). The cytokines used include 100 ng/mL IL-1β (2), 250 ng/mL IL-6 (3), or 500 ng/mL IFN-γ (4). Levels of C3 generated by the cells treated with the cytokines were compared to levels of C3 in untreated cells (1). There was no obvious change in complement C3 expression in 293 and A549 cells treated with any cytokine or in ARPE19 cells treated with 500 ng/mL IFN-γ (4). However, C3 expression was significantly up-regulated in ARPE19 cells treated with 100 ng/mL IL-1β (2) or 250 ng/mL IL-6 (3).

### Example 2

Human Complement C3 siRNAs Suppress IL-1β-Induced Upregulation of Complement C3 Protein in ARPE19 Cells

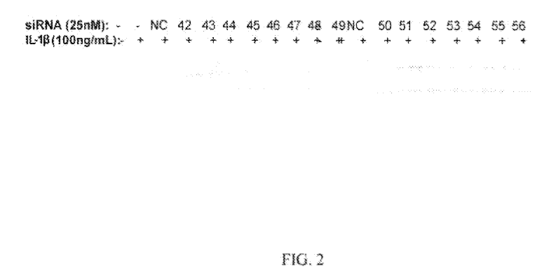

Complement C3 protein expression was upregulated by treating ARPE19 cells with 100 ng/mL of IL-1β treatment. Cells so treated were transfected with 25 nM complement C3 siRNAs or with a negative control siRNA (NC) and C3 protein levels were evaluated by Western blot analysis. As illustrated in FIG. 2, cytokine-induced increase of complement C3 protein levels were significantly reduced in cells transfected with human complement C3 siRNAs SEQ. ID. NOS. 43, 45, 48, 49, 51, 53, and 56. Transfections with the negative control siRNA or mock transfections without siRNA had no effect on complement C3 levels.

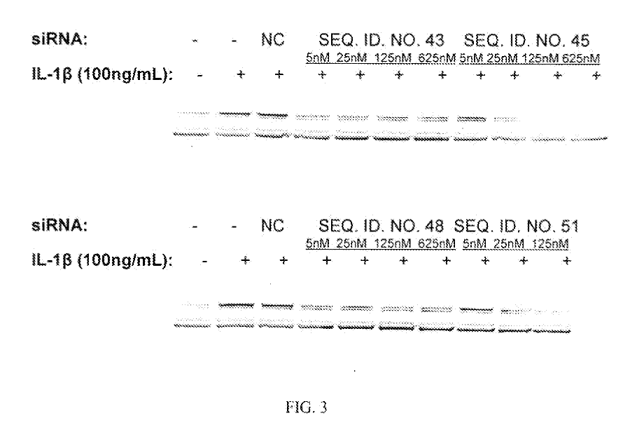

Dose response studies were performed on complement C3 siRNAs (FIG. 3). ARPE19 cells treated with 100 ng/ml IL-1β were transfected with 5 nM, 25 nM, 125 nM or 625 nM complement C3 siRNAs SEQ. ID. NOS. 43, 45, 48, and 51 or a negative control siRNA (NC). C3 protein was then detected by Western blot analysis. Complement C3 siRNAs SEQ. ID. NOS. 45 and 51 showed the most significant dose response.

### Example 3

Cytotoxicity Assay

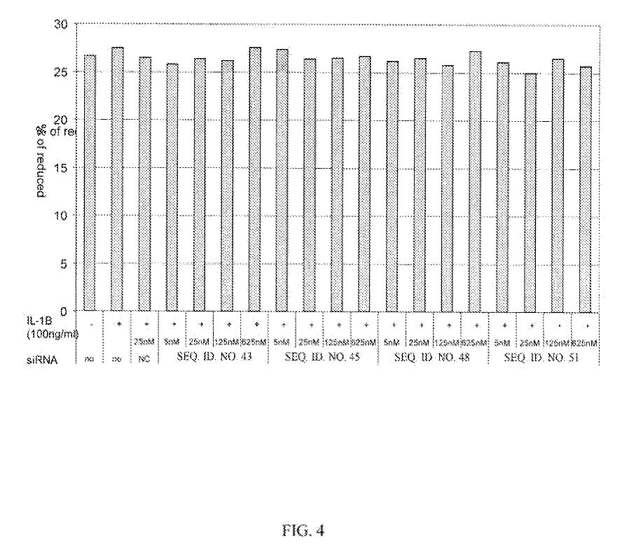

siRNAs were tested for cytotoxicity (FIG. 4). ARPE19 cells treated with 100 ng/mL IL-1β were transfected with 5 nM, 25 nM, 125 nM or 625 nM complement C3 siRNAs or with negative control siRNA (NC). A cytotoxicity assay was performed 48 hours after transfection and IL-1β treatment using alamarBlue™. The siRNAs (5 nM-625 nM) were not cytotoxic to the ARPE19 cells.

Although the present invention has been described in considerable detail with reference to certain preferred embodiments thereof, other versions are possible. Therefore the spirit and scope of the appended claims should not be limited to the description and the preferred versions contained within this specification.

What is claimed is:

1. An isolated siRNA comprising:
a sense RNA strand having a nucleotide sequence substantially identical to a target sequence of from about 19 to about 25 contiguous nucleotides of human C3 protein mRNA; and
an antisense RNA strand having a sequence substantially complementary to the sense RNA strand;
wherein the sense RNA strand and the antisense RNA strand form an RNA duplex.

2. The siRNA of claim 1, wherein the sense RNA strand comprises one RNA molecule, and the antisense RNA strand comprises one RNA molecule.

3. The siRNA of claim 1, wherein the sense and antisense RNA strands of the siRNA are covalently linked.

4. The siRNA of claim 1, wherein the siRNA further comprises non-nucleotide material.

5. The siRNA of claim 1, wherein the sense and antisense RNA strands are stabilized against nuclease degradation.

6. The siRNA of claim 1, wherein the sense RNA strand comprises a first 3′ overhang and/or the antisense RNA strand comprises a second 3′ overhang.

7. The siRNA of claim 6, wherein the first and/or the second 3′ overhang comprises from 1 to about 6 nucleotides.

8. The siRNA of claim 6, wherein the first and/or the second 3′ overhang comprises about 2 nucleotides.

9. The siRNA of claim 6, wherein the dinucleotide comprising the first and/or the second 3′ overhangs is dithymidine (TT) or diuridine (uu).

10. The siRNA of claim 1, wherein sense RNA strand has a sequence corresponding to a sequence selected from SEQ ID NO: 43, SEQ ID NO: 45, SEQ ID NO: 48, SEQ ID NO: 49, SEQ ID NO: 51, SEQ ID NO: 53, and SEQ ID NO: 56.

11. A pharmaceutical composition comprising:
an effective amount of an isolated siRNA having a sense RNA strand including a nucleotide sequence substantially identical to a target sequence of about 19 to about 25 contiguous nucleotides in human C3 protein mRNA and an antisense RNA strand substantially complementary to the sense RNA strand, wherein the sense and an antisense RNA strands form an RNA duplex, and
a pharmaceutically acceptable carrier.

12. The pharmaceutical composition of claim 11, further comprising a delivery reagent.

13. The pharmaceutical composition of claim 11, wherein the delivery agent is selected from lipofectin, lipofectamine, cellfectin, polycations, liposomes, and combinations thereof.

14. The pharmaceutical composition of claim 13, wherein the delivery agent is a liposome.

15. The pharmaceutical composition of claim 13, wherein the liposome further comprises a targeting ligand that targets the liposome to cells at or near the site of a drusen.

16. The pharmaceutical composition of claim 15, wherein the ligand comprises an antibody.

17. The pharmaceutical composition of claim 13, wherein the liposome is modified with an opsonization-inhibition moiety.

18. The pharmaceutical composition of claim 17, wherein the opsonization-inhibiting moiety comprises a PEG, PPG, or derivatives thereof.

19. The pharmaceutical composition of claim 11, wherein the sense RNA strand has a sequence corresponding to a sequence selected from SEQ ID NO: 43, SEQ ID NO: 45, SEQ ID NO: 48, SEQ ID NO: 49, SEQ ID NO: 51, SEQ ID NO: 53, and SEQ ID NO: 56.

20. The pharmaceutical composition of claim 11, wherein the sense RNA strand comprises a first 3′ overhang and/or the antisense RNA strand comprises a second 3′ overhang.

21. The pharmaceutical composition of claim 20, wherein the first and/or the second 3′ overhang comprises from 1 to about 6 nucleotides.

22. The pharmaceutical composition of claim 20, wherein the first and/or the second 3′ overhang comprises about 2 nucleotides.

23. The pharmaceutical composition of claim 20, wherein the first and/or the second 3′ overhangs is a dinucleotide comprising dithymidine (TT) or diuridine (uu).

24. A method for inhibiting expression of human complement system or complement system regulator mRNA comprising:
administering to a subject in need thereof an effective amount an isolated siRNA having a sense RNA strand including a nucleotide sequence substantially identical to a target sequence of about 19 to about 25 contiguous nucleotides in human C3 protein mRNA and an antisense RNA strand substantially complementary to the sense RNA strand, wherein the sense and an antisense RNA strands form an RNA duplex; and
degrading the human C3 regulatory mRNA wherein degrading the human C3 regulatory mRNA inhibits expression of the human C3 regulatory mRNA and inhibits initiation and/or proliferation of a complement system response.

25. The method of claim 24, wherein the subject in need thereof is a human being.

26. The method of claim 24, wherein the effective amount of the siRNA is from about 1 nM to about 100 nM.

27. The method of claim 24, wherein the siRNA is expressed from a recombinant plasmid.

28. The method of claim 24, wherein the siRNA is expressed from a recombinant virus.

29. The method of claim 28, wherein the recombinant virus comprises an adenoviral vector, an adeno-associated viral vector, a lentiviral vector, a retroviral vector, or a herpes virus vector.

30. The method of claim 28, wherein the recombinant viral vector comprises an adeno-associated viral vector.

31. The method of claim 28, wherein the recombinant virus expresses surface proteins from a virus selected from vesicular stomatitis virus, rabies virus, Ebola virus, or Mokola virus.

32. The method of claim 24, wherein the siRNA is administered enterally.

33. The method of claim 32, wherein enterally administered is selected from oral, rectal, and intranasal administration.

34. The method of claim 24, wherein the siRNA is administered parenterally.

35. The method of claim 34, wherein the parenterally administered is selected from intravascular administration, peri- and intra-tissue injection, subcutaneous injection or deposition, or subcutaneous infusion administration, and direct administration at or near a site of drusen formation.

36. The method of claim 35, wherein intravascular administration is selected from intravenous bolus injection, intravenous infusion, intra-arterial bolus injection, intra-arterial infusion and catheter instillation into a vasculature.

37. The method of claim 35, wherein peri- and intra-tissue injection is selected from intra-retinal injection, subretinal injection and intra-vitreal.

38. The method of claim 35, wherein direct administration at or near the site of drusen formation comprises administration by catheter, retinal pellet, suppository, an implant comprising a porous material, an implant comprising a non-porous material, or an implant comprising a gelatinous material.

39. The method of claim 24, wherein the siRNA is administered by eye drops or intraorbital injection.

40. A method of treating a disease in a subject, comprising:
administering to the subject an effective amount of an isolated siRNA having a sense RNA strand including a nucleotide sequence substantially identical to a target sequence of about 19 to about 25 contiguous nucleotides in human C3 protein mRNA and an antisense RNA strand substantially complementary to the sense RNA strand, wherein the sense and an antisense RNA strands form an RNA duplex; and
degrading C3 RNA in the subject.

41. The method of claim 40, wherein the disease is selected from macular degeneration-related disorder, age-related macular disorder, North Carolina macular dystrophy, Sorsby's fundus dystrophy, Stargardt's disease, pattern dystrophy, Best disease, dominant drusen, diabetic retinopathy and malattia leventinese.

42. The method of claim 41, wherein macular degeneration-related disorder is selected from retinal detachment, chorioretinal degenerations, retinal degenerations, photoreceptor degenerations, RPE degenerations, mucopolysaccharidoses, rodcone dystrophies, cone-rod dystrophies, and cone degenerations.

43. The method of claim 40, wherein the disease is age-related macular degeneration.

44. The method of claim 40, wherein the disease is diabetic retinopathy.

45. The method of claim 40, furthering comprising detecting said expression level of the human complement system or complement system regulatory mRNA with urine, blood plasma, serum, whole blood, or eye fluid from the subject.

46. A method of treating an ocular disorder associated with drusen comprising:
ocularly administering to at least one eye of a subject an effective amount of an isolated siRNA having a sense RNA strand including a nucleotide sequence substantially identical to a target sequence of about 19 to about 25 contiguous nucleotides in human C3 protein mRNA and an antisense RNA strand substantially complementary to the sense RNA strand, wherein the sense and an antisense RNA strands form an RNA duplex; and
degrading C3 mRNA in the at least one eye of the subject.

47. The method of claim 46, wherein the ocular disorder is selected from macular degeneration-related disorder, age-related macular disorder, North Carolina macular dystrophy, Sorsby's fundus dystrophy, Stargardt's disease, pattern dystrophy, Best disease, dominant drusen, diabetic retinopathy and malattia leventinese.

48. The method of claim 46, wherein the disease is age-related macular degeneration.

49. The method of claim 46, wherein the disease is diabetic retinopathy.

50. The method of claim 46, wherein the ocular administration is selected from topical, intra-retinal injection, subretinal injection, intravitreal injection, and intraorbital injection.

51. The method of claim 46, wherein the ocular administration is selected from eye drops or intraorbital injection.

52. The method of claim 46, wherein the sense RNA strand has a sequence corresponding to a sequence selected from SEQ ID NO: 43, SEQ ID NO: 45, SEQ ID NO: 48, SEQ ID NO: 49, SEQ ID NO: 51, SEQ ID NO: 53, and SEQ ID NO: 56.

53. The method of claim 46, wherein the siRNA comprises a first 3′ overhang and/or a second 3′ overhang selected from dithymidine (TT) or diuridine (uu).


SEQUENCE LISTING (57 sequences)

SEQ ID NO: 1 - DNA, 2526 bp, Homo sapiens
gcacacagtgcacgaagacgctgtcgggagagcccaggattcaacacgggccttgagaaatgtggctcttgtacctcctggtgccggccctgttctgcagggcaggaggctccattcccatccctcagaagttatttggggaggtgacttcccctctgttccccaagccttaccccaacaactttgaaacaaccactgtgatcacagtccccacgggatacagggtgaagctcgtcttccagcagtttgacctggagccttctgaaggctgcttctatgattatgtcaagatctctgctgataagaaaagcctggggaggttctgtgggcaactgggttctccactgggcaaccccccgggaaagaaggaatttatgtcccaagggaacaagatgctgctgaccttccacacagacttctccaacgaggagaatgggaccatcatgttctacaagggcttcctggcctactaccaagctgtggaccttgatgaatgtgcttcccggagcaaattaggggaggaggatccccagccccagtgccagcacctgtgtcacaactacgttggaggctacttctgttcctgccgtccaggctatgagcttcaggaagacaggcattcctgccaggctgagtgcagcagcgagctgtacacggaggcatcaggctacatctccagcctggagtaccctcggtcctacccccctgacctgcgctgcaactacagcatccgggtggagcggggcctcaccctgcacctcaagttcctggagccttttgatattgatgaccaccagcaagtacactgcccctatgaccagctacagatctatgccaacgggaagaacattggcgagttctgtgggaagcaaaggccccccgacctcgacaccagcagcaatgctgtggatctgctgttcttcacagatgagtcgggggacagccggggctggaagctgcgctacaccaccgagatcatcaagtgcccccagcccaagaccctagacgagttcaccatcatccagaacctgcagcctcagtaccagttccgtgactacttcattgctacctgcaagcaaggctaccagctcatagaggggaaccaggtgctgcattccttcacagctgtctgccaggatgatggcacgtggcatcgtgccatgcccagatgcaagatcaaggactgtgggcagccccgaaacctgcctaatggtgacttccgttacaccaccacaatgggagtgaacacctacaaggcccgtatccagtactactgccatgagccatattacaagatgcagaccagagctggcagcagggagtctgagcaaggggtgtacacctgcacagcacagggcatttggaagaatgaacagaagggagagaagattcctcggtgcttgccagtgtgtgggaagcccgtgaaccccgtggaacagaggcagcgcatcatcggagggcaaaaagccaagatgggcaacttcccctggcaggtgttcaccaacatccacgggcgcgggggcggggccctgctgggcgaccgctggatcctcacagctgcccacaccctgtatcccaaggaacacgaagcgcaaagcaacgcctctttggatgtgttcctgggccacacaaatgtggaagagctcatgaagctaggaaatcaccccatccgcagggtcagcgtccacccggactaccgtcaggatgagtcctacaattttgagggggacatcgccctgctggagctggaaaatagtgtcaccctgggtcccaacctcctccccatctgcctccctgacaacgataccttctacgacctgggcttgatgggctatgtcagtggcttcggggtcatggaggagaagattgctcatgacctcaggtttgtccgtctgcccgtagctaatccacaggcctgtgagaactggctccggggaaagaataggatggatgtgttctctcaaaacatgttctgtgctggacacccatctctaaagcaggacgcctgccagggggatagtgggggcgtttttgcagtaagggacccgaacactgatcgctgggtggccacgggcatcgtgtcctggggcatcgggtgcagcaggggctatggcttctacaccaaagtgctcaactacgtggactggatcaagaaagagatggaggaggaggactgagcccagaattcactaggttcgaatccagagagcagtgtggaaaaaaaaaaacaaaaaacaactgaccagttgttgataaccactaagagtctctattaaaattactgatgcagaaagaccgtgtgtgaaattctctttcctgtagtcccattgatgtactttacctgaaacaacccaaagggcccctttctttcttctgaggattgcagaggatatagttatcaatctctagttgtcactttcctcttccactttgataccattgggtcattgaatataactttttccaaataaagttttatgagaaatgccagtgtgcaaaaaaaaaaaaaaaaaaaaaaaaaaaaaa

SEQ ID NO: 2 - DNA, 2714 bp, Homo sapiens
aatcttatgaacagaaccaggacagggaggctggccggaggttcctgcagagggagcgtcaaggccctgtgctgctgtccctgggggccagaggggttgcccagcatgcccactggcaggagagagggaactgacccacttgctcctaccagcttctgaaggctccaaagtccggaggtgcagaaagccaggaccaagagacaggcagctcaccagggtggacaaatcgccagagatgtggtgcattgtcctgttttcacttttggcatgggtttatgctgagcctaccatgtatggggagatcctgtcccctaactatcctcaggcatatcccagtgaggtagagaaatcttgggacatagaagttcctgaagggtatgggattcacctctacttcacccatctggacattgagctgtcagagaactgtgcgtatgactcagtgcagataatctcaggagacactgaagaagggaggctctgtggacagaggagcagtaacaatccccactctccaattgtggaagagttccaagtcccatacaacaaactccaggtgatctttaagtcagacttttccaatgaagagcgttttacggggtttgctgcatactatgttgccacagacataaatgaatgcacagattttgtagatgtcccttgtagccacttctgcaacaatttcattggtggttacttctgctcctgccccccggaatatttcctccatgatgacatgaagaattgcggagttaattgcagtggggatgtattcactgcactgattggggagattgcaagtcccaattatcccaaaccatatccagagaactcaaggtgtgaataccagatccggttggagaaagggttccaagtggtggtgaccttgcggagagaagattttgatgtggaagcagctgactcagcgggaaactgccttgacagtttagtttttgttgcaggagatcggcaatttggtccttactgtggtcatggattccctgggcctctaaatattgaaaccaagagtaatgctcttgatatcatcttccaaactgatctaacagggcaaaaaaagggctggaaacttcgctatcatggagatccaatgccctgccctaaggaagacactcccaattctgtttgggagcctgcgaaggcaaaatatgtctttagagatgtggtgcagataacctgtctggatgggtttgaagttgtggagggacgtgttggtgcaacatctttctattcgacttgtcaaagcaatggaaagtggagtaattccaaactgaaatgtcaacctgtggactgtggcattcctgaatccattgagaatggtaaagttgaagacccagagagcactttgtttggttctgtcatccgctacacttgtgaggagccatattactacatggaaaatggaggaggtggggagtatcactgtgctggtaacgggagctgggtgaatgaggtgctgggcccggagctgccgaaatgtgttccagtctgtggagtccccagagaaccctttgaagaaaaacagaggataattggaggatccgatgcagatattaaaaacttcccctggcaagtcttctttgacaacccatgggctggtggagcgctcattaatgagtactgggtgctgacggctgctcatgttgtggagggaaacagggagccaacaatgtatgttgggtccacctcagtgcagacctcacggctggcaaaatccaagatgctcactcctgagcatgtgtttattcatccgggatggaagctgctggaagtcccagaaggacgaaccaattttgataatgacattgcactggtgcggctgaaagacccagtgaaaatgggacccaccgtctctcccatctgcctaccaggcacctcttccgactacaacctcatggatggggacctgggactgatctcaggctggggccgaacagagaagagagatcgtgctgttcgcctcaaggcggcaaggttacctgtagctcctttaagaaaatgcaaagaagtgaaagtggagaaacccacagcagatgcagaggcctatgttttcactcctaacatgatctgtgctggaggagagaagggcatggatagctgtaaaggggacagtggtggggcctttgctgtacaggatcccaatgacaagaccaaattctacgcagctggcctggtgtcctgggggccccagtgtgggacctatgggctctacacacgggtaaagaactatgttgactggataatgaagactatgcaggaaaatagcaccccccgtgaggactaatccagatacatcccaccagcctctccaagggtggtgaccaatgcattaccttctgttccttatgatattctcattatttcatcatgactgaaagaagacacgagcgaatgatttaaatagaacttgattgttgagacgccttgctagaggtagagtttgatcatagaattgtgctggtcatacatttgtggtctgactccttggggtcctttccccggagtacctattgtagataacactatgggtggggcactcctttcttgcactattccacagggataccttaattctttgtttcctctttacctgttcaaaattccatttacttgatcattctcagtatccactgtctatgtacaataaaggatgtttataagcaaaaaaaaaaaaaaaaaaaaaaaaaaaaaaaaaa

SEQ ID NO: 3 - DNA, 2609 bp, Homo sapiens
ggctctctacctctcgccgcccctagggaggacaccatgggcccactgatggttcttttttgcctgctgttcctgtacccaggtctggcagactcggctccctcctgccctcagaacgtgaatatctcgggtggcaccttcaccctcagccatggctgggctcctgggagccttctcacctactcctgcccccagggcctgtacccatccccagcatcacggctgtgcaagagcagcggacagtggcagaccccaggagccacccggtctctgtctaaggcggtctgcaaacctgtgcgctgtccagcccctgtctcctttgagaatggcatttataccccacggctggggtcctatcccgtgggtggcaatgtgagcttcgagtgtgaggatggcttcatattgcggggctcgcctgtgcgtcagtgtcgccccaacggcatgtgggatggagaaacagctgtgtgtgataatggggctggccactgccccaacccaggcatttcactgggcgcagtgcggacaggcttccgctttggtcatggggacaaggtccgctatcgctgctcctcgaatcttgtgctcacggggtcttcggagcgggagtgccagggcaacggggtctggagtggaacggagcccatctgccgccaaccctactcttatgacttccctgaggacgtggcccctgccctgggcacttccttctcccacatgcttggggccaccaatcccacccagaagacaaaggaaagcctgggccgtaaaatccaaatccagcgctctggtcatctgaacctctacctgctcctggactgttcgcagagtgtgtcggaaaatgactttctcatcttcaaggagagcgcctccctcatggtggacaggatcttcagctttgagatcaatgtgagcgttgccattatcacctttgcctcagagcccaaagtcctcatgtctgtcctgaacgacaactcccgggatatgactgaggtgatcagcagcctggaaaatgccaactataaagatcatgaaaatggaactgggactaacacctatgcagccttaaacagtgtctatctcatgatgaacaaccaaatgcgactcctcggcatggaaacgatggcctggcaggaaatccgacatgccatcatccttctgacagatggaaagtccaatatgggtggctctcccaagacagctgttgaccatatcagagagatcctgaacatcaaccagaagaggaatgactatctggacatctatgccatcggggtgggcaagctggatgtggactggagagaactgaatgagctagggtccaagaaggatggtgagaggcatgccttcattctgcaggacacaaaggctctgcaccaggtctttgaacatatgctggatgtctccaagctcacagacaccatctgcggggtggggaacatgtcagcaaacgcctctgaccaggagaggacaccctggcatgtcactattaagcccaagagccaagagacctgccggggggccctcatctccgaccaatgggtcctgacagcagctcattgcttccgcgatggcaacgaccactccctgtggagggtcaatgtgggagaccccaaatcccagtggggcaaagaattccttattgagaaggcggtgatctccccagggtttgatgtctttgccaaaaagaaccagggaatcctggagttctatggtgatgacatagctctgctgaagctggcccagaaagtaaagatgtccacccatgccaggcccatctgccttccctgcacgatggaggccaatctggctctgcggagacctcaaggcagcacctgtagggaccatgagaatgaactgctgaacaaacagagtgttcctgctcattttgtcgccttgaatgggagcaaactgaacattaaccttaagatgggagtggagtggacaagctgtgccgaggttgtctcccaagaaaaaaccatgttccccaacttgacagatgtcagggaggtggtgacagaccagttcctatgcagtgggacccaggaggatgagagtccctgcaagggagaatctgggggagcagttttccttgagcggagattcaggttttttcaggtgggtctggtgagctggggtctttacaacccctgccttggctctgctgacaaaaactcccgcaaaagggcccctcgtagcaaggtcccgccgccacgagactttcacatcaatctcttccgcatgcagccctggctgaggcagcacctgggggatgtcctgaattttttacccctctagccatggccactgagccctctgctgccctgccagaatctgccgcccctccatcttctacctctgaatggccacccttagaccctgtgatccatcctctctcctagctgagtaaatccgggtctctaggatgccagaggcagcgcacacaagctgggaaatcctcagggctcctaccagcaggactgcctcgctgccccacctcccgctccttggcctgtccccagattccttccctggttgacttgactcatgcttgtttcactttcacatggaatttcccagttatgaaattaataaaaatcaatggtttccac

SEQ ID NO: 4 - DNA, 5101 bp, Homo sapiens
cactcctccccatcctctccctctgtccctctgtccctctgaccctgcactgtcccagcaccatgggacccacctcaggtcccagcctgctgctcctgctactaacccacctccccctggctctggggagtcccatgtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacgatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacagttctctatcggatcttcaccgtcaaccacaagctgctacccgtgggccggacggtcatggtcaacattgagaacccggaaggcatcccggtcaagcaggactccttgtcttctcagaaccagcttggcgtcttgcccttgtcttgggacattccggaactcgtcaacatgggccagtggaagatccgagcctactatgaaaactcaccacagcaggtcttctccactgagtttgaggtgaaggagtacgtgctgcccagtttcgaggtcatagtggagcctacagagaaattctactacatctataacgagaagggcctggaggtcaccatcaccgccaggttcctctacgggaagaaagtggagggaactgcctttgtcatcttcgggatccaggatggcgaacagaggatttccctgcctgaatccctcaagcgcattccgattgaggatggctcgggggaggttgtgctgagccggaaggtactgctggacggggtgcagaacccccgagcagaagacctggtggggaagtctttgtacgtgtctgccaccgtcatcttgcactcaggcagtgacatggtgcaggcagagcgcagcgggatccccatcgtgacctctccctaccagatccacttcaccaagacacccaagtacttcaaaccaggaatgccctttgacctcatggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctgatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcgctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaagatctgggacgtggtggagaaggcagacatcggctgcaccccgggcagtgggaaggattacgccggtgtcttctccgacgcagggctgaccttcacgagcagcagtggccagcagaccgcccagagggcagaacttcagtgcccgcagccagccgcccgccgacgccgttccgtgcagctcacggagaagcgaatggacaaagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaacctggatgaggacatcattgcagaagagaacatcgtttcccgaagtgagttcccagagagctggctgtggaacgttgaggacttgaaagagccaccgaaaaatggaatctctacgaagctcatgaatatatttttgaaagactccatcaccacgtgggagattctggctgtgagcatgtcggacaagaaagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtgccggaaggaatcagaatgaacaaaactgtggctgttcgcaccctggatccagaacgcctgggccgtgaaggagtgcagaaagaggacatcccacctgcagacctcagtgaccaagtcccggacaccgagtctgagaccagaattctcctgcaagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaaggggtacacccagcagctggccttcagacaacccagctctgcctttgcggccttcgtgaaacgggcacccagcacctggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattggtggattacggaacaacaacgagaaagacatggccctcacggcctttgttctcatctcgctgcaggaggctaaagatatttgcgaggagcaggtcaacagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagagaccaaggaaaatgagggtttcacagtcacagctgaaggaaaaggccaaggcaccttgtcggtggtgacaatgtaccatgctaaggccaaagatcaactcacctgtaataaattcgacctcaaggtcaccataaaaccagcaccggaaacagaaaagaggcctcaggatgccaagaacactatgatccttgagatctgtaccaggtaccggggagaccaggatgccactatgtctatattggacatatccatgatgactggctttgctccagacacagatgacctgaagcagctggccaatggtgttgacagatacatctccaagtatgagctggacaaagccttctccgataggaacaccctcatcatctacctggacaaggtctcacactctgaggatgactgtctagctttcaaagttcaccaatactttaatgtagagcttatccagcctggagcagtcaaggtctacgcctattacaacctggaggaaagctgtacccggttctaccatccggaaaaggaggatggaaagctgaacaagctctgccgtgatgaactgtgccgctgtgctgaggagaattgcttcatacaaaagtcggatgacaaggtcaccctggaagaacggctggacaaggcctgtgagccaggagtggactatgtgtacaagacccgactggtcaaggttcagctgtccaatgactttgacgagtacatcatggccattgagcagaccatcaagtcaggctcggatgaggtgcaggttggacagcagcgcacgttcatcagccccatcaagtgcagagaagccctgaagctggaggagaagaaacactacctcatgtggggtctctcctccgatttctggggagagaagcccaacctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctca

SEQ ID NO: 5 - DNA, 5426 bp, Homo sapiens
agaaggtagcagacagacagacggatctaacctctcttggatcctccagccatgaggctgctctgggggctgatctgggcatccagcttcttcaccttatctctgcagaagcccaggttgctcttgttctctccttctgtggttcatctgggggtccccctatcggtgggggtgcagctccaggatgtgccccgaggacaggtagtgaaaggatcagtgttcctgagaaacccatctcgtaataatgtcccctgctccccaaaggtggacttcacccttagctcagaaagagacttcgcactcctcagtctccaggtgcccttgaaagatgcgaagagctgtggcctccatcaactcctcagaggccctgaggtccagctggtggcccattcgccatggctaaaggactctctgtccagaacgacaaacatccagggtatcaacctgctcttctcctctcgccgggggcacctctttttgcagacggaccagcccatttacaaccctggccagcgggttcggtaccgggtctttgctctggatcagaagatgcgcccgagcactgacaccatcacagtcatggtggagaactctcacggcctccgcgtgcggaagaaggaggtgtacatgccctcgtccatcttccaggatgactttgtgatcccagacatctcagagccagggacctggaagatctcagcccgattctcagatggcctggaatccaacagcagcacccagtttgaggtgaagaaatatgtccttcccaactttgaggtgaagatcacccctggaaagccctacatcctgacggtgccaggccatcttgatgaaatgcagttagacatccaggccaggtacatctatgggaagccagtgcagggggtggcatatgtgcgctttgggctcctagatgaggatggtaagaagactttctttcgggggctggagagtcagaccaagctggtgaatggacagagccacatttccctctcaaaggcagagttccaggacgccctggagaagctgaatatgggcattactgacctccaggggctgcgcctctacgttgctgcagccatcattgagtctccaggtggggagatggaggaggcagagctcacatcctggtattttgtgtcatctcccttctccttggatcttagcaagaccaagcgacaccttgtgcctggggcccccttcctgctgcaggccttggtccgtgagatgtcaggctccccagcttctggcattcctgtcaaagtttctgccacggtgtcttctcctgggtctgttcctgaagtccaggacattcagcaaaacacagacgggagcggccaagtcagcattccaataattatccctcagaccatctcagagctgcagctctcagtatctgcaggctccccacatccagcgatagccaggctcactgtggcagccccaccttcaggaggccccgggtttctgtctattgagcggccggattctcgacctcctcgtgttggggacactctgaacctgaacttgcgagccgtgggcagtggggccaccttttctcattactactacatgatcctatcccgagggcagatcgtgttcatgaatcgagagcccaagaggaccctgacctcggtctcggtgtttgtggaccatcacctggcaccctccttctactttgtggccttctactaccatggagaccacccagtggccaactccctgcgagtggatgtccaggctggggcctgcgagggcaagctggagctcagcgtggacggtgccaagcagtaccggaacggggagtccgtgaagctccacttagaaaccgactccctagccctggtggcgctgggagccttggacacagctctgtatgctgcaggcagcaagtcccacaagcccctcaacatgggcaaggtctttgaagctatgaacagctatgacctcggctgtggtcctgggggtggggacagtgcccttcaggtgttccaggcagcgggcctggccttttctgatggagaccagtggaccttatccagaaagagactaagctgtcccaaggagaagacaacccggaaaaagagaaacgtgaacttccaaaaggcgattaatgagaaattgggtcagtatgcttccccgacagccaagcgctgctgccaggatggggtgacacgtctgcccatgatgcgttcctgcgagcagcgggcagcccgcgtgcagcagccggactgccgggagcccttcctgtcctgctgccaatttgctgagagtctgcgcaagaagagcagggacaagggccaggcgggcctccaacgagccctggagatcctgcaggaggaggacctgattgatgaggatgacattcccgtgcgcagcttcttcccagagaactggctctggagagtggaaacagtggaccgctttcaaatattgacactgtggctccccgactctctgaccacgtgggagatccatggcctgagcctgtccaaaaccaaaggcctatgtgtggccaccccagtccagctccgggtgttccgcgagttccacctgcacctccgcctgcccatgtctgtccgccgctttgagcagctggagctgcggcctgtcctctataactacctggataaaaacctgactgtgagcgtccacgtgtccccagtggaggggctgtgcctggctgggggcggagggctggcccagcaggtgctggtgcctgcgggctctgcccggcctgttgccttctctgtggtgcccacggcagccgccgctgtgtctctgaaggtggtggctcgagggtccttcgaattccctgtgggagatgcggtgtccaaggttctgcagattgagaaggaaggggccatccatagagaggagctggtctatgaactcaaccccttggaccaccgaggccggaccttggaaatacctggcaactctgatcccaatatgatccctgatggggactttaacagctacgtcagggttacagcctcagatccattggacactttaggctctgagggggccttgtcaccaggaggcgtggcctccctcttgaggcttcctcgaggctgtggggagcaaaccatgatctacttggctccgacactggctgcttcccgctacctggacaagacagagcagtggagcacactgcctcccgagaccaaggaccacgccgtggatctgatccagaaaggctacatgcggatccagcagtttcggaaggcggatggttcctatgcggcttggttgtcacgggacagcagcacctggctcacagcctttgtgttgaaggtcctgagtttggcccaggagcaggtaggaggctcgcctgagaaactgcaggagacatctaactggcttctgtcccagcagcaggctgacggctcgttccaggacccctgtccagtgttagacaggagcatgcaggggggtttggtgggcaatgatgagactgtggcactcacagcctttgtgaccatcgcccttcatcatgggctggccgtcttccaggatgagggtgcagagccattgaagcagagagtggaagcctccatctcaaaggcaaactcatttttgggggagaaagcaagtgctgggctcctgggtgcccacgcagctgccatcacggcctatgccctgacactgaccaaggcgcctgtggacctgctcggtgttgcccacaacaacctcatggcaatggcccaggagactggagataacctgtactggggctcagtcactggttctcagagcaatgccgtgtcgcccaccccggctcctcgcaacccatccgaccccatgccccaggccccagccctgtggattgaaaccacagcctacgccctgctgcacctcctgcttcacgagggcaaagcagagatggcagaccaggcttcggcctggctcacccgtcagggcagcttccaagggggattccgcagtacccaagacacggtgattgccctggatgccctgtctgcctactggattgcctcccacaccactgaggagaggggtctcaatgtgactctcagctccacaggccggaatgggttcaagtcccacgcgctgcagctgaacaaccgccagattcgcggcctggaggaggagctgcagttttccttgggcagcaagatcaatgtgaaggtgggaggaaacagcaaaggaaccctgaaggtccttcgtacctacaatgtcctggacatgaagaacacgacctgccaggacctacagatagaagtgacagtcaaaggccacgtcgagtacacgatggaagcaaacgaggactatgaggactatgagtacgatgagcttccagccaaggatgacccagatgcccctctgcagcccgtgacacccctgcagctgtttgagggtcggaggaaccgccgcaggagggaggcgcccaaggtggtggaggagcaggagtccagggtgcactacaccgtgtgcatctggcggaacggcaaggtggggctgtctggcatggccatcgcggacgtcaccctcctgagtggattccacgccctgcgtgctgacctggagaagctgacctccctctctgaccgttacgtgagtcactttgagaccgaggggccccacgtcctgctgtattttgactcggtccccacctcccgggagtgcgtgggctttgaggctgtgcaggaagtgccggtggggctggtgcagccggccagcgcaaccctgtacgactactacaaccccgagcgcagatgttctgtgttttacggggcaccaagtaagagcagactcttggccaccttgtgttctgctgaagtctgccagtgtgctgaggggaagtgccctcgccagcgtcgcgccctggagcggggtctgcaggacgaggatggctacaggatgaagtttgcctgctactacccccgtgtggagtacggcttccaggttaaggttctccgagaagacagcagagctgctttccgcctctttgagaccaagatcacccaagtcctgcacttcaccaaggatgtcaaggccgctgctaatcagatgcgcaacttcctggttcgagcctcctgccgccttcgcttggaacctgggaaagaatatttgatcatgggtctggatggggccacctatgacctcgagggacacccccagtacctgctggactcgaatagctggatcgaggagatgccctctgaacgcctgtgccggagcacccgccagcgggcagcctgtgcccagctcaacgacttcctccaggagtatggcactcaggggtgccaggtgtgagggctgccctcccacctccgctgggaggaacctgaacctgggaaccatgaagctggaagcactgctgtgtccgctttcatgaacacagcctgggaccagggcatattaaaggcttttggcagcaaagtgtcagtgttggc

SEQ ID NO: 6 - DNA, 5426 bp, Homo sapiens
agaaggtagcagacagacagacggatctaacctctcttggatcctccagccatgaggctgctctgggggctgatctgggcatccagcttcttcaccttatctctgcagaagcccaggttgctcttgttctctccttctgtggttcatctgggggtccccctatcggtgggggtgcagctccaggatgtgccccgaggacaggtagtgaaaggatcagtgttcctgagaaacccatctcgtaataatgtcccctgctccccaaaggtggacttcacccttagctcagaaagagacttcgcactcctcagtctccaggtgcccttgaaagatgcgaagagctgtggcctccatcaactcctcagaggccctgaggtccagctggtggcccattcgccatggctaaaggactctctgtccagaacgacaaacatccagggtatcaacctgctcttctcctctcgccgggggcacctctttttgcagacggaccagcccatttacaaccctggccagcgggttcggtaccgggtctttgctctggatcagaagatgcgcccgagcactgacaccatcacagtcatggtggagaactctcacggcctccgcgtgcggaagaaggaggtgtacatgccctcgtccatcttccaggatgactttgtgatcccagacatctcagagccagggacctggaagatctcagcccgattctcagatggcctggaatccaacagcagcacccagtttgaggtgaagaaatatgtccttcccaactttgaggtgaagatcacccctggaaagccctacatcctgacggtgccaggccatcttgatgaaatgcagttagacatccaggccaggtacatctatgggaagccagtgcagggggtggcatatgtgcgctttgggctcctagatgaggatggtaagaagactttctttcgggggctggagagtcagaccaagctggtgaatggacagagccacatttccctctcaaaggcagagttccaggacgccctggagaagctgaatatgggcattactgacctccaggggctgcgcctctacgttgctgcagccatcattgagtctccaggtggggagatggaggaggcagagctcacatcctggtattttgtgtcatctcccttctccttggatcttagcaagaccaagcgacaccttgtgcctggggcccccttcctgctgcaggccttggtccgtgagatgtcaggctccccagcttctggcattcctgtcaaagtttctgccacggtgtcttctcctgggtctgttcctgaagtccaggacattcagcaaaacacagacgggagcggccaagtcagcattccaataattatccctcagaccatctcagagctgcagctctcagtatctgcaggctccccacatccagcgatagccaggctcactgtggcagccccaccttcaggaggccccgggtttctgtctattgagcggccggattctcgacctcctcgtgttggggacactctgaacctgaacttgcgagccgtgggcagtggggccaccttttctcattactactacatgatcctatcccgagggcagatcgtgttcatgaatcgagagcccaagaggaccctgacctcggtctcggtgtttgtggaccatcacctggcaccctccttctactttgtggccttctactaccatggagaccacccagtggccaactccctgcgagtggatgtccaggctggggcctgcgagggcaagctggagctcagcgtggacggtgccaagcagtaccggaacggggagtccgtgaagctccacttagaaaccgactccctagccctggtggcgctgggagccttggacacagctctgtatgctgcaggcagcaagtcccacaagcccctcaacatgggcaaggtctttgaagctatgaacagctatgacctcggctgtggtcctgggggtggggacagtgcccttcaggtgttccaggcagcgggcctggccttttctgatggagaccagtggaccttatccagaaagagactaagctgtcccaaggagaagacaacccggaaaaagagaaacgtgaacttccaaaaggcgattaatgagaaattgggtcagtatgcttccccgacagccaagcgctgctgccaggatggggtgacacgtctgcccatgatgcgttcctgcgagcagcgggcagcccgcgtgcagcagccggactgccgggagcccttcctgtcctgctgccaatttgctgagagtctgcgcaagaagagcagggacaagggccaggcgggcctccaacgagccctggagatcctgcaggaggaggacctgattgatgaggatgacattcccgtgcgcagcttcttcccagagaactggctctggagagtggaaacagtggaccgctttcaaatattgacactgtggctccccgactctctgaccacgtgggagatccatggcctgagcctgtccaaaaccaaaggcctatgtgtggccaccccagtccagctccgggtgttccgcgagttccacctgcacctccgcctgcccatgtctgtccgccgctttgagcagctggagctgcggcctgtcctctataactacctggataaaaacctgactgtgagcgtccacgtgtccccagtggaggggctgtgcctggctgggggcggagggctggcccagcaggtgctggtgcctgcgggctctgcccggcctgttgccttctctgtggtgcccacggcagccaccgctgtgtctctgaaggtggtggctcgagggtccttcgaattccctgtgggagatgcggtgtccaaggttctgcagattgagaaggaaggggccatccatagagaggagctggtctatgaactcaaccccttggaccaccgaggccggaccttggaaatacctggcaactctgatcccaatatgatccctgatggggactttaacagctacgtcagggttacagcctcagatccattggacactttaggctctgagggggccttgtcaccaggaggcgtggcctccctcttgaggcttcctcgaggctgtggggagcaaaccatgatctacttggctccgacactggctgcttcccgctacctggacaagacagagcagtggagcacactgcctcccgagaccaaggaccacgccgtggatctgatccagaaaggctacatgcggatccagcagtttcggaaggcggatggttcctatgcggcttggttgtcacggggcagcagcacctggctcacagcctttgtgttgaaggtcctgagtttggcccaggagcaggtaggaggctcgcctgagaaactgcaggagacatctaactggcttctgtcccagcagcaggctgacggctcgttccaggacctctctccagtgatacataggagcatgcaggggggtttggtgggcaatgatgagactgtggcactcacagcctttgtgaccatcgcccttcatcatgggctggccgtcttccaggatgagggtgcagagccattgaagcagagagtggaagcctccatctcaaaggcaagctcatttttgggggagaaagcaagtgctgggctcctgggtgcccacgcagctgccatcacggcctatgccctgacactgaccaaggcccctgcggacctgcggggtgttgcccacaacaacctcatggcaatggcccaggagactggagataacctgtactggggctcagtcactggttctcagagcaatgccgtgtcgcccaccccggctcctcgcaacccatccgaccccatgccccaggccccagccctgtggattgaaaccacagcctacgccctgctgcacctcctgcttcacgagggcaaagcagagatggcagaccaggctgcggcctggctcacccgtcagggcagcttccaagggggattccgcagtacccaagacacggtgattgccctggatgccctgtctgcctactggattgcctcccacaccactgaggagaggggtctcaatgtgactctcagctccacaggccggaatgggttcaagtcccacgcgctgcagctgaacaaccgccagattcgcggcctggaggaggagctgcagttttccttgggcagcaagatcaatgtgaaggtgggaggaaacagcaaaggaaccctgaaggtccttcgtacctacaatgtcctggacatgaagaacacgacctgccaggacctacagatagaagtgacagtcaaaggccacgtcgagtacacgatggaagcaaacgaggactatgaggactatgagtacgatgagcttccagccaaggatgacccagatgcccctctgcagcccgtgacacccctgcagctgtttgagggtcggaggaaccgccgcaggagggaggcgcccaaggtggtggaggagcaggagtccagggtgcactacaccgtgtgcatctggcggaacggcaaggtggggctgtctggcatggccatcgcggacgtcaccctcctgagtggattccacgccctgcgtgctgacctggagaagctgacctccctctctgaccgttacgtgagtcactttgagaccgaggggccccacgtcctgctgtattttgactcggtccccacctcccgggagtgcgtgggctttgaggctgtgcaggaagtgccggtggggctggtgcagccggccagcgcaaccctgtacgactactacaaccccgagcgcagatgttctgtgttttacggggcaccaagtaagagcagactcttggccaccttgtgttctgctgaagtctgccagtgtgctgaggggaagtgccctcgccagcgtcgcgccctggagcggggtctgcaggacgaggatggctacaggatgaagtttgcctgctactacccccgtgtggagtacggcttccaggttaaggttctccgagaagacagcagagctgctttccgcctctttgagaccaagatcacccaagtcctgcacttcaccaaggatgtcaaggccgctgctaatcagatgcgcaacttcctggttcgagcctcctgccgccttcgcttggaacctgggaaagaatatttgatcatgggtctggatggggccacctatgacctcgagggacacccccagtacctgctggactcgaatagctggatcgaggagatgccctctgaacgcctgtgccggagcacccgccagcgggcagcctgtgcccagctcaacgacttcctccaggagtatggcactcaggggtgccaggtgtgagggctgccctcccacctccgctgggaggaacctgaacctgggaaccatgaagctggaagcactgctgtgtccgctttcatgaacacagcctgggaccagggcatattaaaggcttttggcagcaaagtgtcagtgttggc

SEQ ID NO: 7 - DNA, 5480 bp, Homo sapiens
tatatccgtggtttcctgctacctccaaccatgggccttttgggaatactttgttttttaatcttcctggggaaaacctggggacaggagcaaacatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacaatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcagtaaaagttagagtttattcgttgaatgacgacttgaagccagccaaaagagaaactgtcttaactttcatagatcctgaaggatcagaagttgacatggtagaagaaattgatcatattggaattatctcttttcctgacttcaagattccgtctaatcctagatatggtatgtggacgatcaaggctaaatataaagaggacttttcaacaactggaaccgcatattttgaagttaaagaatatgtcttgccacatttttctgtctcaatcgagccagaatataatttcattggttacaagaactttaagaattttgaaattactataaaagcaagatatttttataataaagtagtcactgaggctgacgtttatatcacatttggaataagagaagacttaaaagatgatcaaaaagaaatgatgcaaacagcaatgcaaaacacaatgttgataaatggaattgctcaagtcacatttgattctgaaacagcagtcaaagaactgtcatactacagtttagaagatttaaacaacaagtacctttatattgctgtaacagtcatagagtctacaggtggattttctgaagaggcagaaatacctggcatcaaatatgtcctctctccctacaaactgaatttggttgctactcctcttttcctgaagcctgggattccatatcccatcaaggtgcaggttaaagattcgcttgaccagttggtaggaggagtcccagtaacactgaatgcacaaacaattgatgtaaaccaagagacatctgacttggatccaagcaaaagtgtaacacgtgttgatgatggagtagcttcctttgtgcttaatctcccatctggagtgacggtgctggagtttaatgtcaaaactgatgctccagatcttccagaagaaaatcaggccagggaaggttaccgagcaatagcatactcatctctcagccaaagttacctttatattgattggactgataaccataaggctttgctagtgggagaacatctgaatattattgttacccccaaaagcccatatattgacaaaataactcactataattacttgattttatccaagggcaaaattatccactttggcacgagggagaaattttcagatgcatcttatcaaagtataaacattccagtaacacagaacatggttccttcatcccgacttctggtctattacatcgtcacaggagaacagacagcagaattagtgtctgattcagtctggttaaatattgaagaaaaatgtggcaaccagctccaggttcatctgtctcctgatgcagatgcatattctccaggccaaactgtgtctcttaatatggcaactggaatggattcctgggtggcattagcagcagtggacagtgctgtgtatggagtccaaagaggagccaaaaagcccttggaaagagtatttcaattcttagagaagagtgatctgggctgtggggcaggtggtggcctcaacaatgccaatgtgttccacctagctggacttaccttcctcactaatgcaaatgcagatgactcccaagaaaatgatgaaccttgtaaagaaattctcaggccaagaagaacgctgcaaaagaagatagaagaaatagctgctaaatataaacattcagtagtgaagaaatgttgttacgatggagcctgcgttaataatgatgaaacctgtgagcagcgagctgcacggattagtttagggccaagatgcatcaaagctttcactgaatgttgtgtcgtcgcaagccagctccgtgctaatatctctcataaagacatgcaattgggaaggctacacatgaagaccctgttaccagtaagcaagccagaaattcggagttattttccagaaagctggttgtgggaagttcatcttgttcccagaagaaaacagttgcagtttgccctacctgattctctaaccacctgggaaattcaaggcgttggcatttcaaacactggtatatgtgttgctgatactgtcaaggcaaaggtgttcaaagatgtcttcctggaaatgaatataccatattctgttgtacgaggagaacagatccaattgaaaggaactgtttacaactataggacttctgggatgcagttctgtgttaaaatgtctgctgtggagggaatctgcacttcggaaagcccagtcattgatcatcagggcacaaagtcctccaaatgtgtgcgccagaaagtagagggctcctccagtcacttggtgacattcactgtgcttcctctggaaattggccttcacaacatcaatttttcactggagacttggtttggaaaagaaatcttagtaaaaacattacgagtggtgccagaaggtgtcaaaagggaaagctattctggtgttactttggatcctaggggtatttatggtaccattagcagacgaaaggagttcccatacaggatacccttagatttggtccccaaaacagaaatcaaaaggattttgagtgtaaaaggactgcttgtaggtgagatcttgtctgcagttctaagtcaggaaggcatcaatatcctaacccacctccccaaagggagtgcagaggcggagctgatgagcgttgtcccagtattctatgtttttcactacctggaaacaggaaatcattggaacatttttcattctgacccattaattgaaaagcagaaactgaagaaaaaattaaaagaagggatgttgagcattatgtcctacagaaatgctgactactcttacagtgtgtggaagggtggaagtgctagcacttggttaacagcttttgctttaagagtacttggacaagtaaataaatacgtagagcagaaccaaaattcaatttgtaattctttattgtggctagttgagaattatcaattagataatggatctttcaaggaaaattcacagtatcaaccaataaaattacagggtaccttgcctgttgaagcccgagagaacagcttatatcttacagcctttactgtgattggaattagaaaggctttcgatatatgccccctggtgaaaatcgacacagctctaattaaagctgacaactttctgcttgaaaatacactgccagcccagagcacctttacattggccatttctgcgtatgctctttccctgggagataaaactcacccacagtttcgttcaattgtttcagctttgaagagagaagctttggttaaaggtaatccacccatttatcgtttttggaaagacaatcttcagcataaagacagctctgtacctaacactggtacggcacgtatggtagaaacaactgcctatgctttactcaccagtctgaacttgaaagatataaattatgttaacccagtcatcaaatggctatcagaagagcagaggtatggaggtggcttttattcaacccaggacacaatcaatgccattgagggcctgacggaatattcactcctggttaaacaactccgcttgagtatggacatcgatgtttcttacaagcataaaggtgccttacataattataaaatgacagacaagaatttccttgggaggccagtagaggtgcttctcaatgatgacctcattgtcagtacaggatttggcagtggcttggctacagtacatgtaacaactgtagttcacaaaaccagtacctctgaggaagtttgcagcttttatttgaaaatcgatactcaggatattgaagcatcccactacagaggctacggaaactctgattacaaacgcatagtagcatgtgccagctacaagcccagcagggaagaatcatcatctggatcctctcatgcggtgatggacatctccttgcctactggaatcagtgcaaatgaagaagacttaaaagcccttgtggaaggggtggatcaactattcactgattaccaaatcaaagatggacatgttattctgcaactgaattcgattccctccagtgatttcctttgtgtacgattccggatatttgaactctttgaagttgggtttctcagtcctgccactttcacagtgtacgaataccacagaccagataaacagtgtaccatgttttatagcacttccaatatcaaaattcagaaagtctgtgaaggagccgcgtgcaagtgtgtagaagctgattgtgggcaaatgcaggaagaattggatctgacaatctctgcagagacaagaaaacaaacagcatgtaaaccagagattgcatatgcttataaagttagcatcacatccatcactgtagaaaatgtttttgtcaagtacaaggcaacccttctggatatctacaaaactggggaagctgttgctgagaaagactctgagattaccttcattaaaaaggtaacctgtactaacgctgagctggtaaaaggaagacagtacttaattatgggtaaagaagccctccagataaaatacaatttcagtttcaggtacatctaccctttagattccttgacctggattgaatactggcctagagacacaacatgttcatcgtgtcaagcatttttagctaatttagatgaatttgccgaagatatctttttaaatggatgctaaaattcctgaagttcagctgcatacagtttgcacttatggactcctgttgttgaagttcgtttttttgttttcttctttttttaaacattcatagctggtcttatttgtaaagctcactttacttagaattagtggcacttgcttttattagagaatgatttcaaatgctgtaactttctgaaataacatggccttggagggcatgaagacagatactcctccaaggttattggacaccggaaacaataaattggaacacctcctcaaacctaccactcaggaatgtttgctggggccgaaagaacagtccattgaaagggagtattacaaaaacatggcctttgcttgaaagaaaataccaaggaacaggaaactgatcattaaagcctgagtttgctttcaaaaaaaaaaaaaaaaaa

SEQ ID NO: 8 - DNA, 3551 bp, Homo sapiens
ttgccttgtgttagctagcaataagaaaagaagctttgtttggattaacatatataccctcttcattctgcatacctattttttccccaataatttgcagcttaggtccgaggacaccacaaactctgcttaaagggcctggaggctctcaaggcatggccagacgctctgtcttgtacttcatcctgctgaatgctctgatcaacaagggccaagcctgcttctgtgatcactatgcatggactcagtggaccagctgctcaaaaacttgcaattctggaacccagagcagacacagacaaatagtagtagataagtactaccaggaaaacttttgtgaacagatttgcagcaagcaggagactagagaatgtaactggcaaagatgccccatcaactgcctcctgggagattttggaccatggtcagactgtgacccttgtattgaaaaacagtctaaagttagatctgtcttgcgtcccagtcagtttgggggacagccatgcactgagcctctggtagcctttcaaccatgcattccatctaagctctgcaaaattgaagaggctgactgcaagaataaatttcgctgtgacagtggccgctgcattgccagaaagttagaatgcaatggagaaaatgactgtggagacaattcagatgaaagggactgtgggaggacaaaggcagtatgcacacggaagtataatcccatccctagtgtacagttgatgggcaatgggtttcattttctggcaggagagcccagaggagaagtccttgataactctttcactggaggaatatgtaaaactgtcaaaagcagtaggacaagtaatccataccgtgttccggccaatctggaaaatgtcggctttgaggtacaaactgcagaagatgacttgaaaacagatttctacaaggatttaacttctcttggacacaatgaaaatcaacaaggctcattctcaagtcagggggggagctctttcagtgtaccaattttttattcctcaaagagaagtgaaaatatcaaccataattctgccttcaaacaagccattcaagcctctcacaaaaaggattctagttttattaggatccataaagtgatgaaagtcttaaacttcacaacgaaagctaaagatctgcacctttctgatgtctttttgaaagcacttaaccatctgcctctagaatacaactctgctttgtacagccgaatattcgatgactttgggactcattacttcacctctggctccctgggaggcgtgtatgaccttctctatcagtttagcagtgaggaactaaagaactcaggtttaaccgaggaagaagccaaacactgtgtcaggattgaaacaaagaaacgcgttttatttgctaagaaaacaaaagtggaacataggtgcaccaccaacaagctgtcagagaaacatgaaggttcatttatacagggagcagagaaatccatatccctgattcgaggtggaaggagtgaatatggagcagctttggcatgggagaaagggagctctggtctggaggagaagacattttctgagtggttagaatcagtgaaggaaaatcctgctgtgattgactttgagcttgcccccatcgtggacttggtaagaaacatcccctgtgcagtgacaaaacggaacaacctcaggaaagctttgcaagagtatgcagccaagttcgatccttgccagtgtgctccatgccctaataatggccgacccaccctctcagggactgaatgtctgtgtgtgtgtcagagtggcacctatggtgagaactgtgagaaacagtctccagattataaatccaatgcagtagacggacagtggggttgttggtcttcctggagtacctgtgatgctacttataagagatcgagaacccgagaatgcaataatcctgccccccaacgaggagggaaacgctgtgagggggagaagcgacaagaggaagactgcacattttcaatcatggaaaacaatggacaaccatgtatcaatgatgatgaagaaatgaaagaggtcgatcttcctgagatagaagcagattccgggtgtcctcagccagttcctccagaaaatggatttatccggaatgaaaagcaactatacttggttggagaagatgttgaaatttcatgccttactggctttgaaactgttggataccagtacttcagatgcttaccagacgggacctggagacaaggggatgtggaatgccaacggacggagtgcatcaagccagttgtgcaggaagtcctgacaattacaccatttcagagattgtatagaattggtgaatccattgagctaacttgccccaaaggctttgttgttgctgggccatcaaggtacacatgccaggggaattcctggacaccacccatttcaaactctctcacctgtgaaaaagatactctaacaaaattaaaaggccattgtcagctgggacagaaacaatcaggatctgaatgcatttgtatgtctccagaagaagactgtagccatcattcagaagatctctgtgtgtttgacacagactccaacgattactttacttcacccgcttgtaagtttttggctgagaaatgtttaaataatcagcaactccattttctacatattggttcctgccaagacggccgccagttagaatggggtcttgaaaggacaagactttcatccaacagcacaaagaaagaatcctgtggctatgacacctgctatgactgggaaaaatgttcagcctccacttccaaatgtgtctgcctattgcccccacagtgcttcaagggtggaaaccaactctactgtgtcaaaatgggatcatcaacaagtgagaaaacattgaacatctgtgaagtgggaactataagatgtgcaaacaggaagatggaaatactgcatcctggaaagtgtttggcctagcacaattactgctaggcccagcacaatgaacagatttaccatcccgaagaaccaactcctacaaatgagaattcttgcacaaacagcagactggcatgctcaaagttactgacaaaaattattttctgttagtttgagatcattattctcccctgactctcctgtttgggcatgtcttattcagttccagctcatgacgccctgtagcatacccctaggtaccaacttccacagcagtctcgtaaattctcctgttcacattgtacaaaaataatgtgacttctgaggcccttatgtagcctgtgacattaagcattctcacaattagaaataagaataaaacccataattttcttcaatgagttaataaacagaaatctccagaacctctgaaacacattcttgaagcccagctttcatatcttcattcaacaaataatttctgagtgtgtatacaggatgtcaagtactgaccaaagtcctgagaactcggcagataataaaacagacaaaagcctttgccttcatgaagcatacattcattcaggggtagacacacaaaaaatgaaataaacaggtaaaatatgtagc

SEQ ID NO: 9 - DNA, 4034 bp, Homo sapiens
agggagaggcagagaggcaggcagcctgctgggctcttcctgctgttgaaaacttacccggcccttacagaggaaatcttcctcctctcttctgccctgaatgttttcccaaacatgaaggtgataagcttattcattttggtgggatttataggagagttccaaagtttttcaagtgcctcctctccagtcaactgccagtgggacttctatgccccttggtcagaatgcaatggctgtaccaagactcagactcgcaggcggtcagttgctgtgtatgggcagtatggaggccagccttgtgttggaaatgcttttgaaacacagtcctgtgaacctacaagaggatgtccaacagaggagggatgtggagagcgtttcaggtgcttttcaggtcagtgcatcagcaaatcattggtttgcaatggggattctgactgtgatgaagacagtgctgatgaagacagatgtgaggactcagaaaggagaccttcctgtgatatcgataaacctcctcctaacatagaacttactggaaatggttacaatgaactcactggccagtttaggaacagagtcatcaataccaaaagttttggtggtcaatgtagaaaggtgtttagtggggatggaaaagatttctacaggctgagtggaaatgtcctgtcctatacattccaggtgaaaataaataatgattttaattatgaattttacaatagtacttggtcttatgtaaaacatacgtcgacagaacacacatcatctagtcggaagcgctccttttttagatcttcatcatcttcttcacgcagttatacttcacataccaatgaaatccataaaggaaagagttaccaactgctggttgttgagaacactgttgaagtggctcagttcattaataacaatccagaatttttacaacttgctgagccattctggaaggagctttcccacctcccctctctgtatgactacagtgcctaccgaagattaatcgaccagtacgggacacattatctgcaatctgggtcgttaggaggagaatacagagttctattttatgtggactcagaaaaattaaaacaaaatgattttaattcagtcgaagaaaagaaatgtaaatcctcaggttggcattttgtcgttaaattttcaagtcatggatgcaaggaactggaaaacgctttaaaagctgcttcaggaacccagaacaatgtattgcgaggagaaccgttcatcagagggggaggtgcaggcttcatatctggccttagttacctagagctggacaatcctgctggaaacaaaaggcgatattctgcctgggcagaatctgtgactaatcttcctcaagtcataaaacaaaagctgacacctttatatgagctggtaaaggaagtaccttgtgcctctgtgaaaaaactatacctgaaatgggctcttgaagagtatctggatgaatttgacccctgtcattgccggccttgtcaaaatggtggtttggctactgttgaggggacccattgtctgtgccattgcaaaccgtacacatttggtgcggcgtgtgagcaaggagtcctcgtagggaatcaagcaggaggggttgatggaggttggagttgctggtcctcttggagcccctgtgtccaagggaagaaaacaagaagccgtgaatgcaataacccacctcccagtgggggtgggagatcctgcgttggagaaacgacagaaagcacacaatgcgaagatgaggagctggagcacttgaggttgcttgaaccacattgctttcctttgtctttggttccaacagaattctgtccatcacctcctgccttgaaagatggatttgttcaagatgaaggtacaatgtttcctgtggggaaaaatgtagtgtacacttgcaatgaaggatactctcttattggaaacccagtggccagatgtggagaagatttacggtggcttgttggggaaatgcattgtcagaaaattgcctgtgttctacctgtactgatggatggcatacagagtcacccccaaaaacctttctacacagttggtgagaaggtgactgtttcctgttcaggtggcatgtccttagaaggtccttcagcatttctctgtggctccagccttaagtggagtcctgagatgaagaatgcccgctgtgtacaaaaagaaaatccgttaacacaggcagtgcctaaatgtcagcgctgggagaaactgcagaattcaagatgtgtttgtaaaatgccctacgaatgtggaccttccttggatgtatgtgctcaagatgagagaagcaaaaggatactgcctctgacagtttgcaagatgcatgttctccactgtcagggtagaaattacacccttactggtagggacagctgtactctgcctgcctcagctgagaaagcttgtggtgcctgcccactgtggggaaaatgtgatgctgagagcagcaaatgtgtctgccgagaagcatcggagtgcgaggaagaagggtttagcatttgtgtggaagtgaacggcaaggagcagacgatgtctgagtgtgaggcgggcgctctgagatgcagagggcagagcatctctgtcaccagcataaggccttgtgctgcggaaacccagtaggctcctggaggccctggtcagcttgcttggaatccagcaggcagctggggctgagtgaaaacatctgcacaactgggcactggacagcttttccttcttctccagtgtctaccttcctcctcaactcccagccatctgtataaacacaatcctttgttctcccaaatctgaatcgaattactcttttgcctcctttttaatgtcagtaaggatatgagcctttgcacaggctggctgcgtgttcttgaaataggtgttaccttctctgggccttggttttttaaaatctgtaaaattagaggattgcactagagaaacttgaatgctccattcaggcctatcattttattaagtatgattgacacagcccatgggccagaacacactctacaaaatgactaggataacagaaagaacgtgatctcctgattagagagggtggttttcctcaatggaaccaaatataaagaggacttgaacaaaaatgacagatacaaactatttctatcctgagtagtaatctcacacttcatcctatagagtcaaccaccacagataggaattccttattctttttttaatttttttaagacagagtctcactttgttgcccaggctggagcgcagtggggtgatctcatctccctgcaacctccgcctcctgggttcaagcgattcttgtgcctcagcttcccaagcagctgggattacaggtgcccgccaccacgcccagctaatttttgcatttttagtagagatggggtttcaccatgttggccacgctcgtctccaactcctgacctcaggtaatccgcctgccttggcctcccaaagtgctgggattacagacatgaaccaccacgcctggctggaatacttactcttgtcgggagattgaaccactaaaatgttagagcagaattcattatgctgtggtcacaggggtgtcttgtctgagaacaaatacaattcagtcttctctttggggttttagtatgtgtcaaacataggactggaagtttgcccctgttcttttttcttttgaaagaacatcagttcatgcctgaggcatgagtgactgtgcatttgagaatagttttccctattctgtggatacagtcccagagttttcagggagtacacaggtagattagtttgaagcattgaccttttatttattccttatttctctttcatcaaaacaaaacagcagctgtgggaggagaaatgagagggcttaaatgaaatttaaaataagctatattatacaaatactatctctgtattgttctgaccctggtaaatatatttcaaaacttcagatgacaaggattagaacactcattaaagatgctattcttcagaaaaaaaaaaaaaaaaaaaaaaaa

SEQ ID NO: 10 - DNA, 2397 bp, Homo sapiens
ttttttttttcatcctactttgttttattgggcgttgattgttacaggtcccagcctgtagacatcttttactccaatttcctgaatagatagctttattccttcaaggtaatatagtgcggtggcttctggctgagatgtttgctgttgttttcttcatcttgtctttgatgacttgtcagcctggggtaactgcacaggagaaggtgaaccagagagtaagacgggcagctacacccgcagcagttacctgccagctgagcaactggtcagagtggacagattgctttccgtgccaggacaaaaagtaccgacaccggagcctcttgcagccaaacaagtttgggggaaccatctgcagtggtgacatctgggatcaagccagctgctccagttctacaacttgtgtaaggcaagcacagtgtggacaggatttccagtgtaaggagacaggtcgctgcctgaaacgccaccttgtgtgtaatggagaccaggactgccttgatggctctgatgaggacgactgtgaagatgtcagggccattgacgaagactgcagccagtatgaaccaattccaggatcacagaaggcagccttggggtacaatatcctgacccaggaagatgctcagagtgtgtacgatgccagttattatgggggccagtgtgagacggtatacaatggggaatggagggagcttcgatatgactccacctgtgaacgtctctactatggagatgatgagaaatactttcggaaaccctacaactttctgaagtaccactttgaagccctggcagatactggaatctcctcagagttttatgataatgcaaatgaccttctttccaaagttaaaaaagacaagtctgactcatttggagtgaccatcggcataggcccagccggcagccctttattggtgggtgtaggtgtatcccactcacaagacacttcattcttgaacgaattaaacaagtataatgagaagaaattcattttcacaagaatcttcacaaaggtgcagactgcacattttaagatgaggaaggatgacattatgctggatgaaggaatgctgcagtcattaatggagcttccagatcagtacaattatggcatgtatgccaagttcatcaatgactatggcacccattacatcacatctggatccatgggtggcatttatgaatatatcctggtgattgacaaagcaaaaatggaatcccttggtattaccagcagagatatcacgacatgttttggaggctccttgggcattcaatatgaagacaaaataaatgttggtggaggtttatcaggagaccattgtaaaaaatttggaggtggcaaaactgaaagggccaggaaggccatggctgtggaagacattatttctcgggtgcgaggtggcagttctggctggagcggtggcttggcacagaacaggagcaccattacataccgttcctgggggaggtcattaaagtataatcctgttgttatcgattttgagatgcagcctatccacgaggtgctgcggcacacaagcctggggcctctggaggccaagcgccagaacctgcgccgcgccttggaccagtatctgatggaattcaatgcctgccgatgtgggccttgcttcaacaatggggtgcccatcctcgagggcaccagctgcaggtgccagtgccgcctgggtagcttgggtgctgcctgtgagcaaacacagacagaaggagccaaagcagatgggagctggagttgctggagctcctggtctgtatgcagagcaggcatccaggaaaggagaagagagtgtgacaatccagcacctcagaatggaggggcctcgtgtccagggcggaaagtacagacgcaggcttgctgagggcctctggacacaggctggaccagatgctgtggatgtcgacccctgcactgactattggataaagacttctttcaactaagagaagatgcaaatcagcacacttttttctttgttctgccagcttccaggcctaagactaggttttgctgtctacagccaactattctattagttacaaaactcaatcattttattcagcaactggatgttgactgttaactagaagctctgtcctacttacagcactttggatcatcaaaaaaataaagtaaaatagaaaactgagaaaactcaatccatgaccagggagaacttacaggatgttagagacaaaacaagcagacacctgaaacaatcaacgcccaataaaacaaagtaggatgaaaattctcttagttctttgataacaatttgttcactcatagaaacattattaattggtagggtaagcagacactctgaaacaatgagaaaaatactaaaaattgacttgagttatttc

SEQ ID NO: 11 - DNA, 1173 bp, Homo sapiens
gtgtctcagccacagcggcttcaccatgcacagctgggagcgcctggcagttctggtcctcctaggagcggccgcctgcgcggcgccgccccgtggtcggatcctgggcggcagagaggccgaggcgcacgcgcggccctacatggcgtcggtgcagctgaacggcgcgcacctgtgcggcggcgtcctggtggcggagcagtgggtgctgagcgcggcgcactgcctggaggacgcggccgacgggaaggtgcaggttctcctgggcgcgcactccctgtcgcagccggagccctccaagcgcctgtacgacgtgctccgcgcagtgccccacccggacagccagcccgacaccatcgaccacgacctcctgctgctacagctgtcggagaaggccacactgggccctgctgtgcgccccctgccctggcagcgcgtggaccgcgacgtggcaccgggaactctctgcgacgtggccggctggggcatagtcaaccacgcgggccgccgcccggacagcctgcagcacgtgctcttgccagtgctggaccgcgccacctgcaaccggcgcacgcaccacgacggcgccatcaccgagcgcttgatgtgcgcggagagcaatcgccgggacagctgcaagggtgactccgggggcccgctggtgtgcgggggcgtgctcgagggcgtggtcacctcgggctcgcgcgtttgcggcaaccgcaagaagcccgggatctacacccgcgtggcgagctatgcggcctggatcgacagcgtcctggcctagggtgccggggcctgaaggtcagggtcacccaagcaacaaagtcccgagcaatgaagtcatccactcctgcatctggttggtctttattgagcacctactatatgcagaaggggaggccgaggtgggaggatcattggatctcaggagttcgagatcagcatgggccacgtagcgcgactccatctctacaaataaataaaaaattagctgggcaattggcgggcatggaggtgggtgcttgtagttccagctactcaggaggctgaggtgggaggatgacttgaacgcaggaggctgaggctgcagtgagttgtgattgcaccactgccctccagcctgggcaacagagtgaaaccttgtctctctctacaaaaaaaaaaaaaaaa

SEQ ID NO: 12 - DNA, 857 bp, Homo sapiens
ctgggactttggtggtgctacccttggcctcccagagtcctgccaccctgctgccgccaccatgctgccccctgggactgcgaccctcttgactctgctcctggcagctggctcgctgggccagaagcctcagaggccacgccggcccgcatcccccatcagcaccatccagcccaaggccaattttgatgctcagcagtttgcagggacctggctccttgtggctgtgggctccgcttgccgtttcctgcaggagcagggccaccgggccgaggccaccacactgcatgtggctccccagggcacagccatggctgtcagtaccttccgaaagctggatgggatctgctggcaggtgcgccagctctatggagacacaggggtcctcggccgcttcctgcttcaagcccgaggcgcccgaggggctgtgcacgtggttgtcgctgagaccgactaccagagtttcgctgtcctgtacctggagcgggcggggcagctgtcagtgaagctctacgcccgctcgctccctgtgagcgactcggtcctgagtgggtttgagcagcgggtccaggaggcccacctgactgaggaccagatcttctacttccccaagtacggcttctgcgaggctgcagaccagttccacgtcctggacgaagtgaggaggtgaggccggcacacagctccagtgctgagaagtcagtgccccgagagacgaccccaccagtggggtgcccgctgcctgtcctccgtgaaaccagcctcagatcagggccctgccacccagggcaggggatcttctgccggctgccccagaggacagtgggtggagtggtacctacttattaaatgtctcc

SEQ ID NO: 13 - DNA, 2094 bp, Homo sapiens
gcttgttccctgtcctctggccctttgcaaataaatgccttaccagacctgccctgccaccccactcgcagccacccagcaagagcagcatgtcagcctgccggagctttgcagttgcaatctgcattttagaaataagcatcctcacagcacagtacacgaccagttatgacccagagctaacagaaagcagtggctctgcatcacacatagactgcagaatgagcccctggagtgaatggtcacaatgcgatccttgtctcagacaaatgtttcgttcaagaagcattgaggtctttggacaatttaatgggaaaagatgcaccgacgctgtgggagacagacgacagtgtgtgcccacagagccctgtgaggatgctgaggatgactgcggaaatgactttcaatgcagtacaggcagatgcataaagatgcgacttcggtgtaatggtgacaatgactgcggagacttttcagatgaggatgattgtgaaagtgagccccgtcccccctgcagagacagagtggtagaagagtctgagctggcacgaacagcaggctatgggatcaacattttagggatggatcccctaagcacaccttttgacaatgagttctacaatggactctgtaaccgggatcgggatggaaacactctgacatactaccgaagaccttggaacgtggcttctttgatctatgaaaccaaaggcgagaaaaatttcagaaccgaacattacgaagaacaaattgaagcatttaaaagtatcatccaagagaagacatcaaattttaatgcagctatatctctaaaatttacacccactgaaacaaataaagctgaacaatgttgtgaggaaacagcctcctcaatttctttacatggcaagggtagttttcggttttcatattccaaaaatgaaacttaccaactatttttgtcatattcttcaaagaaggaaaaaatgtttctgcatgtgaaaggagaaattcatctgggaagatttgtaatgagaaatcgcgatgttgtgctcacaacaacttttgtggatgatataaaagctttgccaactacctatgaaaagggagaatattttgcctttttggaaacctatggaactcactacagtagctctgggtctctaggaggactctatgaactaatatatgttttggataaagcttccatgaagcggaaaggtgttgaactaaaagacataaagagatgccttgggtatcatctggatgtatctctggctttctctgaaatctctgttggagctgaatttaataaagatgattgtgtaaagaggggagagggtagagctgtaaacatcaccagtgaaaacctcatagatgatgttgtttcactcataagaggtggaaccagaaaatatgcatttgaactgaaagaaaagcttctccgaggaaccgtgattgatgtgactgactttgtcaactgggcctcttccataaatgatgctcctgttctcattagtcaaaaactgtctcctatatataatctggttccagtgaaaatgaaaaatgcacacctaaagaaacaaaacttggaaagagccattgaagactatatcaatgaatttagtgtaagaaaatgccacacatgccaaaatggaggtacagtgattctaatggatggaaagtgtttgtgtgcctgcccattcaaatttgagggaattgcctgtgaaatcagtaaacaaaaaatttctgaaggattgccagccctagagttccccaatgaaaaatagagctgttggcttctctgagctccagtggaagaagaaaacactagtaccttcagatcctacccctgaagataatcttagctgccaagtaaatagcaacatgcttcatgaaaatcctaccaacctctgaagtctcttctctcttaggtctataattttttttttaatttttcttccttaaactcctgtgatgtttccattttttgttccctaatgagaagtcaacagtgaaatacgccagaactgctttatcccacggaaaatgccaatctcttctaaaaaaaaacaaaattaaactaaaaaaaaaaaaaaaaaaaaaaaaaaaaaa

SEQ ID NO: 14 - DNA, 2574 bp, Homo sapiens
ttggatgttccgggaaagtgatgtgggtaggacaggcggggcgagccgcaggtgccagaacacagattgtataaaaggctgggggctggtggggagcaggggaagggaatgtgaccaggtctaggtctggagtttcagcttggacactgagccaagcagacaagcaaagcaagccaggacacaccatcctgccccaggcccagcttctctcctgccttccaacgccatggggagcaatctcagcccccaactctgcctgatgccctttatcttgggcctcttgtctggaggtgtgaccaccactccatggtctttggcccggccccagggatcctgctctctggagggggtagagatcaaaggcggctccttccgacttctccaagagggccaggcactggagtacgtgtgtccttctggcttctacccgtaccctgtgcagacacgtacctgcagatctacggggtcctggagcaccctgaagactcaagaccaaaagactgtcaggaaggcagagtgcagagcaatccactgtccaagaccacacgacttcgagaacggggaatactggccccggtctccctactacaatgtgagtgatgagatctctttccactgctatgacggttacactctccggggctctgccaatcgcacctgccaagtgaatggccgatggagtgggcagacagcgatctgtgacaacggagcggggtactgctccaacccgggcatccccattggcacaaggaaggtgggcagccagtaccgccttgaagacagcgtcacctaccactgcagccgggggcttaccctgcgtggctcccagcggcgaacgtgtcaggaaggtggctcttggagcgggacggagccttcctgccaagactccttcatgtacgacacccctcaagaggtggccgaagctttcctgtcttccctgacagagaccatagaaggagtcgatgctgaggatgggcacggcccaggggaacaacagaagcggaagatcgtcctggacccttcaggctccatgaacatctacctggtgctagatggatcagacagcattggggccagcaacttcacaggagccaaaaagtgtctagtcaacttaattgagaaggtggcaagttatggtgtgaagccaagatatggtctagtgacatatgccacataccccaaaatttgggtcaaagtgtctgaagcagacagcagtaatgcagactgggtcacgaagcagctcaatgaaatcaattatgaagaccacaagttgaagtcagggactaacaccaagaaggccctccaggcagtgtacagcatgatgagctggccagatgacgtccctcctgaaggctggaaccgcacccgccatgtcatcatcctcatgactgatggattgcacaacatgggcggggacccaattactgtcattgatgagatccgggacttgctatacattggcaaggatcgcaaaaacccaagggaggattatctggatgtctatgtgtttggggtcgggcctttggtgaaccaagtgaacatcaatgctttggcttccaagaaagacaatgagcaacatgtgttcaaagtcaaggatatggaaaacctggaagatgttttctaccaaatgatcgatgaaagccagtctctgagtctctgtggcatggtttgggaacacaggaagggtaccgattaccacaagcaaccatggcaggccaagatctcagtcattcgcccttcaaagggacacgagagctgtatgggggctgtggtgtctgagtactttgtgctgacagcagcacattgtttcactgtggatgacaaggaacactcaatcaaggtcagcgtaggaggggagaagcgggacctggagatagaagtagtcctatttcaccccaactacaacattaatgggaaaaaagaagcaggaattcctgaattttatgactatgacgttgccctgatcaagctcaagaataagctgaaatatggccagactatcaggcccatttgtctcccctgcaccgagggaacaactcgagctttgaggcttcctccaactaccacttgccagcaacaaaaggaagagctgctccctgcacaggatatcaaagctctgtttgtgtctgaggaggagaaaaagctgactcggaaggaggtctacatcaagaatggggataagaaaggcagctgtgagagagatgctcaatatgccccaggctatgacaaagtcaaggacatctcagaggtggtcacccctcggttcctttgtactggaggagtgagtccctatgctgaccccaatacttgcagaggtgattctggcggccccttgatagttcacaagagaagtcgtttcattcaagttggtgtaatcagctggggagtagtggatgtctgcaaaaaccagaagcggcaaaagcaggtacctgctcacgcccgagactttcacatcaacctctttcaagtgctgccctggctgaaggagaaactccaagatgaggatttgggttttctataaggggtttcctgctggacaggggcgtgggattgaattaaaacagctgcgacaac

SEQ ID NO: 15 - DNA, 1173 bp, Homo sapiens; identical to SEQ ID NO: 11

SEQ ID NO: 16 - DNA, 1043 bp, Homo sapiens
tgttaatgaaagcagattcaaagcaacaccaccaccactgaagtatttttagttatataagattggaactaccaagcatgtggctcctggtcagtgtaattctaatctcacggatatcctctgttgggggagaagcaatgttctgtgattttccaaaaataaaccatggaattctatatgatgaagaaaaatataagccattttcccaagttcctacaggggaagttttctattactcctgtgaatataattttgtgtctccttcaaaatccttttggactcgcataacgtgcgcagaagaaggatggtcaccaacaccaaagtgtctcagactgtgtttctttccttttgtggaaaatggtcattctgaatcttcaggacaaacacatctggaaggtgatactgtacaaattatttgcaacacaggatacagacttcaaaacaatgagaacaacatttcatgtgtagaacggggctggtccactcctcccaaatgcaggtccactatttctgcagaaaaatgtgggccccctccacctattgacaatggagacattacttcattcctgttgtcagtatatgctccaggttcatcagttgagtaccagtgccagaacttgtatcaacttgagggtaacaatcaaataacatgtagaaacggacaatggtcagaaccaccaaaatgcttagatccatgtgtaatatcacaagaaattatggaaaaatataacataaaattaaagtggacaaaccaacaaaagctttattcaagaacaggtgacatagttgaatttgtttgtaaatctggatatcatccaacaaaatctcattcatttcgagcaatgtgtcagaatgggaaactggtatatcccagttgtgaagaaaaatagaatcaatggcattactattagtaaaatgcacacctttttctgaatttactattatatttgttttcaatttcatttttcaagtactgttttactcatttttattcataaataaagttttgtgttgatttgtgaaaatgcaattacaagagccaa

SEQ ID NO: 17 - DNA, 1292 bp, Homo sapiens
tgaaagatttcaaaccccaaacagtgcaactgaaacttttgcattactatactactgagaatatctaacatgttgttactaatcaatgtcattctgaccttgtgggtttcctgtgctaatggacaagaagtgaaaccttgtgattttccagaaattcaacatggaggtctatattataagagtttgcgtagactatactttccagcagctgcaggacaatcttattcctattactgtgatcaaaattttgtgactccttcaggaagttactgggattacattcattgcacacaagatggttggtcaccaacggtcccatgcctcagaacatgctcaaaatcagatatagaaattgaaaatggattcatttctgaatcttcctctatttatattttaaataaagaaatacaatataaatgtaaaccaggatatgcaacagcagatggaaattcttcaggttcaattacatgtttgcaaaatggatggtcagcacaaccaatttgcattaaattttgtgatatgcctgtttttgagaattccagagccaagagtaatggcatgcggtttaagctccatgacacattggactacgaatgctacgatggatatgaaatcagttatggaaacaccacaggttccatagtgtgtggtgaagatgggtggtcccatttcccaacatgttataattcttcagaaaagtgtgggcctcctccacctattagcaatggtgataccacctcctttctactaaaagtgtatgtgccacagtcaagagtcgagtaccaatgccagtcctactatgaacttcagggttctaattatgtaacatgtagtaatggagagtggtcggaaccaccaagatgcatacatccatgtataataactgaagaaaacatgaataaaaataacatacagttaaaaggaaaaagtgacataaaatattatgcaaaaacaggggataccattgaatttatgtgtaaattgggatataatgcgaatacatcagttctatcatttcaagcagtgtgtagggaaggcatagtggaataccccagatgcgaataaggcagcattgttaccctaaatgtatgtccaacttccacttctcactcttatggtctcaaagcttgcaaagatagcttctgatattgttgtaatttctactttatttcaaagaaaattaatataatagtttcaatttgcaacttaatatgttctcaaaaatatgttaaaacaaactaaattattgcttatgcttgtactaaaataataaaaactacccttatattgga

SEQ ID NO: 18 - DNA, 2823 bp, Homo sapiens
ggcaggtgcttgttactgttaatgaaagcagatttaaagcaacaccaccatcactggagtatttttagttatatacgattgagactaccaagcatgttgctcttattcagtgtaatcctaatctcatgggtatccactgttgggggagaaggaacactttgtgattttccaaaaatacaccatggatttctgtatgatgaagaagattataaccctttttcccaagttcctacaggggaagttttctattactcctgtgaatataattttgtgtctccttcaaaatccttttggactcgcataacatgcacagaagaaggatggtcaccaacaccgaagtgtctcagaatgtgttcctttccttttgtgaaaaatggtcattctgaatcttcaggactaatacatctggaaggtgatactgtacaaattatttgcaacacaggatacagccttcaaaacaatgagaaaaacatttcgtgtgtagaacggggctggtccactcctcccatatgcagcttcactaaaggagaatgtcatgttccaattttagaagccaatgtagatgctcagccaaaaaaagaaagctacaaagttggagacgtgttgaaattctcctgcagaaaaaatcttataagagttggatcagactcagttcaatgttaccaatttgggtggtcacctaactttccaacatgcaaaggacaagtacgatcatgtggtccacctcctcaactctccaatggtgaagttaaggagataagaaaagaggaatatggacacaatgaagtagtggaatatgattgcaatcctaattttataataaacgggcctaagaaaatacaatgtgtggatggagaatggacaactttacccacttgtgttgaacaagtgaaaacatgtggatacatacctgaactcgagtacggttatgttcagccgtctgtccctccctatcaacatggagtttcagtcgaggtgaattgcagaaatgaatatgcaatgattggaaataacatgattacctgtattaatggaatatggacagagcttcctatgtgtgttgcaacacaccaacttaagaggtgcaaaatagcaggagttaatataaaaacattactcaagctatctgggaaagaatttaatcataattctagaatacgttacagatgttcagacatcttcagatacaggcactcagtctgtataaacgggaaatggaatcctgaagtagactgcacagaaaaaagggaacaattctgcccaccgccacctcagatacctaatgctcagaatatgacaaccacagtgaattatcaggatggagaaaaagtagctgttctctgtaaagaaaactatctacttccagaagcaaaagaaattgtatgtaaagatggacgatggcaatcattaccacgctgtgttgagtctactgcatattgtgggccccctccatctattaacaatggagataccacctcattcccattatcagtatatcctccagggtcaacagtgacgtaccgttgccagtccttctataaactccagggctctgtaactgtaacatgcagaaataaacagtggtcagaaccaccaagatgcctagatccatgtgtggtatctgaagaaaacatgaacaaaaataacatacagttaaaatggagaaacgatggaaaactctatgcaaaaacaggggatgctgttgaattccagtgtaaattcccacataaagcgatgatatcatcaccaccatttcgagcaatctgtcaggaagggaaatttgaatatcctatatgtgaatgaagcaagcataattttcctgaatatattcttcaaacatccatctacgctaaaagtagccattatgtagccaattctgtagttacttcttttattctttcaggtgttgtttaactcagttttatttagaactctggatttttagagctttagaaatttgtaagctgagagaacaatgtttcacttaataggagggtgtcttagtccatattacattgttataacagagtatcacagactggataacttctaaccaatagtttatttgtttcataaatctaaaagctgagaagtccaagatggtggggctgcctctggtgagggtcttctcgaagcatcataatatgctggaaggcatcacaacatggtggaagggatcacgtggcaaaagagcatgtacatgggagtgagagaaaaagagagagagagacagagtggcgggggccggggaggagcgcaaactcatcctttataaagacaccactcctgagataacaatccaatcccatgataatgacattaatccattcaagaagatagagctctcgtgacttaatcaccttctaaagatctcacctgacaacactgttgcattggcagttaagtttccacgtaaactttcggggacacattcaaaccacaggagaaactcaaattgttcctgggcaaatcacaacatggggaattttattcataaatgtccacagaaacagtaaatgttctcgcttcagaacttaattcatctaatccctcctgtttgtctcaaattataggataactttgaaactttctgaattaacgttatttaaaaggaaatgtagatgttattttagtctctatcttcaggttattatcacttaaaaacctgcgaaagctgtcaacttttgtggttgtagcaagtattaataaatatttataaatcctctaatgtaagtctagctacctatccaatactaaataccccttaaagtattaaatgcactatctgctgtaaacggaaaaaaaaaaaaaaaaaaaaaaaaa

SEQ ID NO: 19 - DNA, 2160 bp, Homo sapiens
ctttaatgggaaggccattttttggtttcagttacggtaaatctctctggatttcagccaaattctttcagagttcaaaagtacaaagctctttaggaggtttgttcttttgaaacattttttcagaggttaaaaattaaatttcaaaagaatacctggagtggaaaagagttctcagcagagacaaagaccccgaacacctccaacatgaagcttcttcatgttttcctgttatttctgtgcttccacttaaggttttgcaaggtcacttatacatctcaagaggatctggtggagaaaaagtgcttagcaaaaaaatatactcacctctcctgcgataaagtcttctgccagccatggcagagatgcattgagggcacctgtgtttgtaaactaccgtatcagtgcccaaagaatggcactgcagtgtgtgcaactaacaggagaagcttcccaacatactgtcaacaaaagagtttggaatgtcttcatccagggacaaagtttttaaataacggaacatgcacagccgaaggaaagtttagtgtttccttgaagcatggaaatacagattcagagggaatagttgaagtaaaacttgtggaccaagataagacaatgttcatatgcaaaagcagctggagcatgagggaagccaacgtggcctgccttgaccttgggtttcaacaaggtgctgatactcaaagaaggtttaagttgtctgatctctctataaattccactgaatgtctacatgtgcattgccgaggattagagaccagtttggctgaatgtacttttactaagagaagaactatgggttaccaggatttcgctgatgtggtttgttatacacagaaagcagattctccaatggatgacttctttcagtgtgtgaatgggaaatacatttctcagatgaaagcctgtgatggtatcaatgattgtggagaccaaagtgatgaactgtgttgtaaagcatgccaaggcaaaggcttccattgcaaatcgggtgtttgcattccaagccagtatcaatgcaatggtgaggtggactgcattacaggggaagatgaagttggctgtgcaggctttgcatctgtggctcaagaagaaacagaaattttgactgctgacatggatgcagaaagaagacggataaaatcattattacctaaactatcttgtggagttaaaaacagaatgcacattcgaaggaaacgaattgtgggaggaaagcgagcacaactgggagacctcccatggcaggtggcaattaaggatgccagtggaatcacctgtgggggaatttatattggtggctgttggattctgactgctgcacattgtctcagagccagtaaaactcatcgttaccaaatatggacaacagtagtagactggatacaccccgaccttaaacgtatagtaattgaatacgtggatagaattattttccatgaaaactacaatgcaggcacttaccaaaatgacatcgctttgattgaaatgaaaaaagacggaaacaaaaaagattgtgagctgcctcgttccatccctgcctgtgtcccctggtctccttacctattccaacctaatgatacatgcatcgtttctggctggggacgagaaaaagataacgaaagagtcttttcacttcagtggggtgaagttaaactaataagcaactgctctaagttttacggaaatcgtttctatgaaaaagaaatggaatgtgcaggtacatatgatggttccatcgatgcctgtaaaggggactctggaggccccttagtctgtatggatgccaacaatgtgacttatgtctggggtgttgtgagttggggggaaaactgtggaaaaccagagttcccaggtgtttacaccaaagtggccaattattttgactggattagctaccatgtaggaaggccttttatttctcagtacaatgtataaaattgtgatctctctcttcattctattctttttctctcaagagttccatttaatggaaataaaacggtataattaataattctctaggggggaaaaatgaagcaaatctcactggatatttttaaaggtctccacagagtttatgccatattggaattttgttgtataattctcaaataaatattttggtgaagcatacaa

SEQ ID NO: 20 - DNA, 1678 bp, Homo sapiens
gagcaaacagagcagcagaaaaggcagttcctcttctccagtgccctccttccctgtctctgcctctccctcccttcctcaggcatcagagcggagacttcagggagaccagagcccagcttgccaggcactgagctagaagccctgccatggcacccctgagaccccttctcatactggccctgctggcatgggttgctctggctgaccaagagtcatgcaagggccgctgcactgagggcttcaacgtggacaagaagtgccagtgtgacgagctctgctcttactaccagagctgctgcacagactatacggctgagtgcaagccccaagtgactcgcggggatgtgttcactatgccggaggatgagtacacggtctatgacgatggcgaggagaaaaacaatgccactgtccatgaacaggtggggggcccctccctgacctctgacctccaggcccagtccaaagggaatcctgagcagacacctgttctgaaacctgaggaagaggcccctgcgcctgaggtgggcgcctctaagcctgaggggatagactcaaggcctgagacccttcatccagggagacctcagcccccagcagaggaggagctgtgcagtgggaagcccttcgacgccttcaccgacctcaagaacggttccctctttgccttccgagggcagtactgctatgaactggacgaaaaggcagtgaggcctgggtaccccaagctcatccgagatgtctggggcatcgagggccccatcgatgccgccttcacccgcatcaactgtcaggggaagacctacctcttcaagggtagtcagtactggcgctttgaggatggtgtcctggaccctgattacccccgaaatatctctgacggcttcgatggcatcccggacaacgtggatgcagccttggccctccctgcccatagctacagtggccgggagcgggtctacttcttcaaggggaaacagtactgggagtaccagttccagcaccagcccagtcaggaggagtgtgaaggcagctccctgtcggctgtgtttgaacactttgccatgatgcagcgggacagctgggaggacatcttcgagcttctcttctggggcagaacctctgctggtaccagacagccccagttcattagccgggactggcacggtgtgccagggcaagtggacgcagccatggctggccgcatctacatctcaggcatggcaccccgcccctccttggccaagaaacaaaggtttaggcatcgcaaccgcaaaggctaccgttcacaacgaggccacagccgtggccgcaaccagaactcccgccggccatcccgcgccacgtggctgtccttgttctccagtgaggagagcaacttgggagccaacaactatgatgactacaggatggactggcttgtgcctgccacctgtgaacccatccagagtgtcttcttcttctctggagacaagtactaccgagtcaatcttcgcacacggcgagtggacactgtggaccctccctacccacgctccatcgctcagtactggctgggctgcccagctcctggccatctgtaggagtcagagcccacatggccgggccctctgtagctccctcctcccatctccttcccccagcccaataaaggtcccttagccccgagtttaaa

SEQ ID NO: 21 - DNA, 1681 bp, Homo sapiens
gccgtccaagggtccattggttgccatagagatcgtcgagcgctgggcctgtgatcgctgaggggcgagcagttgcgaccctgggctcctggggacctgagcgttatgtctttccgcgacctccgcaatttcacagagatgatgagagccctgggataccctcgacatatttctatggaaaatttccgtacacccaattttggacttgtatctgaagtgcttctctggcttgtgaaaagatatgagccccagactgacatcccgcctgacgtggatactgaacaggaccgagttttcttcattaaggcaattgcccagttcatggccaccaaggcacatataaaactcaacactaagaagctttatcaagcagatgggtatgcggtaaaagagctgctgaagatcacatctgtcctttataatgctatgaagaccaaggggatggagggctctgaaatagtagaggaagatgtcaacaagttcaagtttgatcttggctcaaagattgcagatttgaaggcagccaggcagcttgcgtctgaaatcacctccaaaggagcatctctgtatgacttgctcggcatggaagtagagttgagggaaatgagaacagaagccattgccagacctctggaaataaacgagactgaaaaagtgatgagaattgcaataaaagagattttgacacaggttcagaagactaaagacctgctcaataatgtggcctctgatgaagctaatttagaagccaaaatcgaaaagagaaaattagaactggaaagaaatcggaagcgactagagactctgcagagtgtcaggccatgttttatggatgagtatgagaagactgaggaagaattacaaaagcagtatgacacttatctggagaaatttcaaaatctgacttatctggaacaacagcttgaagaccatcataggatggagcaagaaaggtttgaggaagctaaaaacactctctgcctgatacagaacaagctcaaggaggaagagaagcgcctgctcaagagtggaagtaacgatgactcggacatagacatccaggaggacgatgaatccgacagtgagttggaagaaaggcggctgcccaagccacagacagccatggagatgctcatgcaaggaagacctggcaaacgcattgtgggcacgatgcaaggtggagactccgatgacaatgaggactcggaggagagtgaaattgacatggaagatgatgatgacgaggatgacgatttggaagacgagagcatttctctctcaccaaccaagcccaatcgaagggtccggaaatctgaacccctggatgagagtgacaatgacttctgacccttttgccaagggaccctggcagattaaaaccctcagacttgtaggtaaatgggaacttagaaggttaggaaggtaacccctgttttgtttactaagctggctggactcatgatcactgaagcaatacttatttctgctttagcctcctatgtttgcattccatgaagcttaaataagaattgaagcaaatccctaagatttatttttttccaccttatttatcttctaaaacttgaggaatgcatgtgttcttagtgattcacatccacgggacaaaaactcaagaagaaataagagctgacgccacacaaaaaaaaaaaaaaaaaaaaaa

SEQ ID NO: 22 - DNA, 2859 bp, Homo sapiens
ctttccgcggcattctttgggcgtgagtcatgcaggtttgcagccagccccaaagggggtgtgtgcgcgagcagagcgctataaatacggcgcctcccagtgcccacaacgcggcgtcgccaggaggagcgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaactaaaatatattgctgcaaatggaagcttttctgttctattataaaactgtcgatgtattctgaccaaggtgcgacaatctcctaaaggaatacactgaaagttaaggagaagaatcagtaagtgtaaggtgtacttggtattataatgcataattgatgttttcgttatgaaaacatttggtgcccagaagtccaaattatcagttttatttgtaagagctattgcttttgcagcggttttatttgtaaaagctgttgatttcgagttgtaagagctcagcatcccaggggcatcttcttgactgtggcatttcctgtccaccgccggtttatatgatcttcatacctttccctggaccacaggcgtttctcggcttttagtctgaaccatagctgggctgcagtaccctacgctgccagcaggtggccatgactacccgtggtaccaatctcagtcttaaagctcaggcttttcgttcattaacattctctgatagaattctggtcatcagatgtactgcaatggaacaaaactcatctggctgcatcccaggtgtgtagcaaagtccacatgtaaatttatagcttagaatattcttaagtcactgtcccttgtctctctttgaagttataaacaacaaacttaaagcttagcttatgtccaaggtaagtattttagcatggctgtcaaggaaattcagagtaaagtcagtgtgattcacttaatgatatacattaattagaattatggggtcagaggtatttgcttaagtgatcataattgtaaagtatatgtcacattgtcacattaatgtcaaaaaaaaaaaaaaaaaa

SEQ ID NO: 23 - DNA, 2979 bp, Homo sapiens
gggcagcctgctgtcggcttagaggggatgggcagtgtggagggcctggcagagcaagaggactcatccttccaaagggactttctctgggaagcctgctcctcgggccactgcgaaccctctctactctccgaagggaattgtccttcctggcttccactacttccacccctgaatgcacaggcagcccggcccaagtctcccactagggatgcagatggattcggtgtgaagggctggctgctgttgcctccggctcttgaaagtcaagttcagaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaactaaaatatattgctgcaaatggaagcttttctgttctattataaaactgtcgatgtattctgaccaaggtgcgacaatctcctaaaggaatacactgaaagttaaggagaagaatcagtaagtgtaaggtgtacttggtattataatgcataattgatgttttcgttatgaaaacatttggtgcccagaagtccaaattatcagttttatttgtaagagctattgcttttgcagcggttttatttgtaaaagctgttgatttcgagttgtaagagctcagcatcccaggggcatcttcttgactgtggcatttcctgtccaccgccggtttatatgatcttcatacctttccctggaccacaggcgtttctcggcttttagtctgaaccatagctgggctgcagtaccctacgctgccagcaggtggccatgactacccgtggtaccaatctcagtcttaaagctcaggcttttcgttcattaacattctctgatagaattctggtcatcagatgtactgcaatggaacaaaactcatctggctgcatcccaggtgtgtagcaaagtccacatgtaaatttatagcttagaatattcttaagtcactgtcccttgtctctctttgaagttataaacaacaaacttaaagcttagcttatgtccaaggtaagtattttagcatggctgtcaaggaaattcagagtaaagtcagtgtgattcacttaatgatatacattaattagaattatggggtcagaggtatttgcttaagtgatcataattgtaaagtatatgtcacattgtcacattaatgtcaaaaaaaaaaaaaaaaaa

SEQ ID NO: 24 - DNA, 2256 bp, Homo sapiens
tgaaattcagggactctttggtggagcaattaccagtcaacttcagggtattatgataaactctgatctggggaggaaccaggactacatagatcaaggcagttttcttctttgagaaactatcccagatatcatcatagagtcttctgctcttcctcaactaccaaagaaaaacatcagcgaagcagcaggccatgcaccccccaaaaactccatctggggctcttcatagaaaaaggaaaatggcagcctggcccttctccaggctgtggaaagtctctgatccaattctcttccaaatgaccttgatcgctgctctgttgcctgctgttcttggcaattgtggtcctccacccactttatcatttgctgccccgatggatattacgttgactgagacacgcttcaaaactggaactactctgaaatacacctgcctccctggctacgtcagatcccattcaactcagacgcttacctgtaattctgatggcgaatgggtgtataacaccttctgtatctacaaacgatgcagacacccaggagagttacgtaatgggcaagtagagattaagacagatttatcttttggatcacaaatagaattcagctgttcagaaggatttttcttaattggctcaaccactagtcgttgtgaagtccaagatagaggagttggctggagtcatcctctcccacaatgtgaaattgtcaagtgtaagcctcctccagacatcaggaatggaaggcacagcggtgaagaaaatttctacgcatacggcttttctgtcacctacagctgtgacccccgcttctcactcttgggccatgcctccatttcttgcactgtggagaatgaaacaataggtgtttggagaccaagccctcctacctgtgaaaaaatcacctgtcgcaagccagatgtttcacatggggaaatggtctctggatttggacccatctataattacaaagacactattgtgtttaagtgccaaaaaggttttgttctcagaggcagcagtgtaattcattgtgatgctgatagcaaatggaatccttctcctcctgcttgtgagcccaatagttgtattaatttaccagacattccacatgcttcctgggaaacatatcctaggccgacaaaagaggatgtgtatgttgttgggactgtgttaaggtaccgctgtcatcctggctacaaacccactacagatgagcctacgactgtgatttgtcagaaaaatttgagatggaccccataccaaggatgtgaggcgttatgttgccctgaaccaaagctaaataatggtgaaatcactcaacacaggaaaagtcgtcctgccaatcactgtgtttatttctatggagatgagatttcattttcatgtcatgagaccagtaggttttcagctatatgccaaggagatggcacgtggagtccccgaacaccatcatgtggagacatttgcaattttcctcctaaaattgcccatgggcattataaacaatctagttcatacagctttttcaaagaagagattatatatgaatgtgataaaggctacattctggtcggacaggcgaaactctcctgcagttattcacactggtcagctccagcccctcaatgtaaagctctgtgtcggaaaccagaattagtgaatggaaggttgtctgtggataaggatcagtatgttgagcctgaaaatgtcaccatccaatgtgattctggctatggtgtggttggtccccaaagtatcacttgctctgggaacagaacctggtacccagaggtgcccaagtgtgagtgggagacccccgaaggctgtgaacaagtgctcacaggcaaaagactcatgcagtgtctcccaaacccagaggatgtgaaaatggccctggaggtatataagctgtctctggaaattgaacaactggaactacagagagacagcgcaagacaatccactttggataaagaactataatttttctcaaaagaaggaggaaaaggtgtcttgctggcttgcctcttgcaattcaatacagatcagtttagcaaatctactgtcaatttggcagtgatattcatcataataaatatctagaaatgataatttgctaaagtttagtgctttgagattgtgaaattattaatcatcctctgtgtggctcatgtttttgcttttcaacacacaaagcacaaattttttttcgattaaaaatgtatgtataaaaaactaaaaaaaaaaaaaa

SEQ ID NO: 25 - DNA, 848 bp, Homo sapiens
agcctggggagaggactttgatcaccagatgtttttttggtgtgcgtgctgtcttatggttgcgtggcgagtttctgcttcagatgcagagcactgtccagagcttcctccagtggacaatagcatatttgtcgcaaaggaggtggaaggacagattctggggacttacgtttgtatcaagggctaccacctggtaggaaagaagacccttttttgcaatgcctctaaggagtgggataacaccactactgagtgccgcttgggccactgtcctgatcctgtgctggtgaatggagagttcagttcttcagggcctgtgaatgtaagtgacaaaatcacgtttatgtgcaatgaccactacatcctcaagggcagcaatcggagccagtgtctagaggaccacacctgggcacctccctttcccatctgcaaaagtagggactgtgaccctcctgggaatccagttcatggctattttgaaggaaataacttcaccttaggatccaccattagttattactgtgaagacaggtactacttagtgggcgtgcaggagcagcaatgcgttgatggggagtggagcagtgcacttccagtctgcaagttgatccaggaagctcccaaaccagagtgtgagaaggcacttcttgcctttcaggagagtaagaacctctgcgaagccatggagaactttatgcaacaattaaaggaaagtggcatgacaatggaggagctaaaatattctctggagctgaagaaagctgagttgaaggcaaaattgttgtaacactacagctgagcagatgtaatagaaataaacctatgaataaattttcttcttggttctg

SEQ ID NO: 26 - DNA, 2017 bp, Homo sapiens
ccgagcgtgcccgcggcgctgcccctcctcggggagctgccccggctgctgctgctggtgctgttgtgcctgccggccgtgtggggtgactgtggccttcccccagatgtacctaatgcccagccagctttggaaggccgtacaagttttcccgaggatactgtaataacgtacaaatgtgaagaaagctttgtgaaaattcctggcgagaaggactcagtgacctgccttaagggcatgcaatggtcagatattgaagagttctgcaatcgtagctgcgaggtgccaacaaggctaaattctgcatccctcaaacagccttatatcactcagaattattttccagtcggtactgttgtggaatatgagtgccgtccaggttacagaagagaaccttctctatcaccaaaactaacttgccttcagaatttaaaatggtccacagcagtcgaattttgtaaaaagaaatcatgccctaatccgggagaaatacgaaatggtcagattgatgtaccaggtggcatattatttggtgcaaccatctccttctcatgtaacacagggtacaaattatttggctcgacttctagtttttgtcttatttcaggcagctctgtccagtggagtgacccgttgccagagtgcagagaaatttattgtccagcaccaccacaaattgacaatggaataattcaaggggaacgtgaccattatggatatagacagtctgtaacgtatgcatgtaataaaggattcaccatgattggagagcactctatttattgtactgtgaataatgatgaaggagagtggagtggcccaccacctgaatgcagaggaaaatctctaacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgggcacacgtgtttcacgttgacaggtttgcttgggacgctagtaaccatgggcttgctgacttagccaaagaagagttaagaagaaaatacacacaagtatacagactgttcctagtttcttagacttatctgcatattggataaaataaatgcaattgtgctcttcatttaggatgctttcattgtctttaagatgtgttaggaatgtcaacagagcaaggagaaaaaaggcagtcctggaatcacattcttagcacacctgcgcctcttgaaaatagaacaacttgcagaattgagagtgattcctttcctaaaagtgtaagaaagcatagagatttgttcgtattaagaatgggatcacgaggaaaagagaaggaaagtgatttttttccacaagatctgaaatgatatttccacttataaaggaaataaaaaatgaaaaacattatttggatatcaaaagcaaataaaaacccaattcagtctcttctaagcaaaattgctaaagagagatgaccacattataaagtaatctttggctaaggcattttcatctttccttcggttggcaaaatattttaaaggtaaaacatgctggtgaaccagggtgttgatggtgataagggaggaatatagaatgaaagactgaatcttcctttgttgcacaaatagagtttggaaaaagcctgtgaaaggtgtcttctttgacttaatgtctttaaaagtatccagagatactacaatattaacataagaaaagattatatattatttctgaatcgagatgtccatagtcaaatttgtaaatcttattcttttgtaatatttatttatatttatttatgacagtgaacattctgattttacatgtaaaacaagaaaagttgaagaagatatgtgaagaaaaatgtatttttcctaaatagaaataaatgatcccattttttggt

SEQ ID NO: 27 - DNA, 2017 bp, Homo sapiens; identical to SEQ ID NO: 26

SEQ ID NO: 28 - DNA, 21 bp, Homo sapiens
aatccgagccgttctctacaa

SEQ ID NO: 29 - DNA, 21 bp, Homo sapiens
aacaagctctgccgtgatgaa

SEQ ID NO: 30 - DNA, 21 bp, Homo sapiens
aatggacaaagtcggcaagta

SEQ ID NO: 31 - DNA, 21 bp, Homo sapiens
aactacatgaacctacagaga

SEQ ID NO: 32 - DNA, 21 bp, Homo sapiens
aaaaagcggccagtcagaaga

SEQ ID NO: 33 - DNA, 21 bp, Homo sapiens
aatgattggtggattacggaa

SEQ ID NO: 34 - DNA, 21 bp, Homo sapiens
aagtcctcgttgtccgttcca

SEQ ID NO: 35 - DNA, 21 bp, Homo sapiens
aaatgattggtggattacgga

SEQ ID NO: 36 - DNA, 21 bp, Homo sapiens
aattaccggcagaaccaagag

SEQ ID NO: 37 - DNA, 21 bp, Homo sapiens
aaatggaatctctacgaagct

SEQ ID NO: 38 - DNA, 21 bp, Homo sapiens
aatgaacagagatactacggt

SEQ ID NO: 39 - DNA, 21 bp, Homo sapiens
aagccttggctcaataccaaa

SEQ ID NO: 40 - DNA, 21 bp, Homo sapiens
aagcgcattccgattgaggat

SEQ ID NO: 41 - DNA, 21 bp, Homo sapiens
aatggaatctctacgaagctc

SEQ ID NO: 42 - RNA, 21 nt, Homo sapiens
aauccgagccguucucuacaa

SEQ ID NO: 43 - RNA, 21 nt, Homo sapiens
aacaagcucugccgugaugaa

SEQ ID NO: 44 - RNA, 21 nt, Homo sapiens
aauggacaaagucggcaagua

SEQ ID NO: 45 - RNA, 21 nt, Homo sapiens
aacuacaugaaccuacagaga

SEQ ID NO: 46 - RNA, 21 nt, Homo sapiens
aaaaagcggccagucagaaga

SEQ ID NO: 47 - RNA, 21 nt, Homo sapiens
aaugauugguggauuacggaa

SEQ ID NO: 48 - RNA, 21 nt, Homo sapiens
aaguccucguuguccguucca

SEQ ID NO: 49 - RNA, 21 nt, Homo sapiens
aaaugauugguggauuacgga

SEQ ID NO: 50 - RNA, 21 nt, Homo sapiens
aauuaccggcagaaccaagag

SEQ ID NO: 51 - RNA, 21 nt, Homo sapiens
aaauggaaucucuacgaagcu

SEQ ID NO: 52 - RNA, 21 nt, Homo sapiens
aaugaacagagauacuacggu

SEQ ID NO: 53 - RNA, 21 nt, Homo sapiens
aagccuuggcucaauaccaaa

SEQ ID NO: 54 - RNA, 21 nt, Homo sapiens
aagcgcauuccgauugaggau

SEQ ID NO: 55 - RNA, 21 nt, Homo sapiens
aauggaaucucuacgaagcuc

SEQ ID NO: 56 - RNA, 21 nt, Homo sapiens
aaccucaucgccaucgacucc

SEQ ID NO: 57 - RNA, 21 nt, Homo sapiens
ggagucgauggcgaugagguu